(19) United States
(12) Patent Application Publication
(10) Pub. No.: US 2011/0097344 A1
(43) Pub. Date: Apr. 28, 2011

(54) Compositions, Methods for Treatment, and Diagnoses of Autoimmunity-Related Disorders and Methods for Making Such Compositions

(75) Inventors: Oleg DARASHKEVICH, Minsk (BY); Stuart Juckett, Amagansett, NY (US)

(73) Assignee: EIGER HEALTH PARTNERS, LLC., Amagansett, NY (US)

(21) Appl. No.: 12/910,452

(22) Filed: Oct. 22, 2010

Related U.S. Application Data
(60) Provisional application No. 61/254,072, filed on Oct. 22, 2009.
(60) Provisional application No. 61/306,718, filed on Feb. 22, 2010.

(51) Int. Cl. A61K 39/00; C07K 1/36; C07K 16/18; G01N 30/02; A61P 35/00; A61P 37/00; A61P 25/00; A61P 9/00; A61P 11/00; A61P 13/02; A61P 1/00; A61P 5/00; A61P 37/06; A61P 17/00; A61P 31/00; A61P 35/02

(52) U.S. Cl. 424/176.1; 530/390.1; 530/387.1; 436/161

(57) ABSTRACT

The present invention provides compositions and methods useful in the diagnosis and treatment of autoimmunity-related disorders, including cancers and other disorders involving angiogenesis, as well as non-cancer disorders involving a dysfunction in the immune system. In some embodiments, the invention described a plasma assay. In other embodiments, urine assay. In certain other embodiments, the invention provides therapeutic methods comprising removing toxic autoantibodies from the circulation of a patient, e.g., via plasmapheresis, and subsequently infusing the patient with one or more immunoglobulins or immunoglobulin complexes to restore the immune system of the patient to a baseline status whereby the patient's restored immune system either eliminates the source of the disorder (e.g., in the case of cancers) or no longer causes the disease or disorder (e.g., in the case of autoimmune disorders such as multiple sclerosis, psoriasis, latent autoimmune type 1 diabetes in adults (LADA) and the like). Methods of making the high activity IVIG preparation are also provided.

### CROSS REFERENCE TO RELATED APPLICATIONS

This application claims the benefit of U.S. provisional application No. 61/254,072, filed Oct. 22, 2009, and 61/306,718, filed Feb. 22, 2010, both of which are incorporated herein by reference in their entirety.

### BACKGROUND OF THE INVENTION

1. Field of the Invention

The present invention is in the fields of medicine, immunology and pharmacology, particularly in the areas of medical therapeutics and diagnostics. More particularly, the present invention provides compositions and methods useful in the treatment of diseases and disorders, particularly autoimmunity-related diseases and disorders, including cancers and other disorders involving autoimmune-related angiogenesis, as well as non -cancer disorders involving a dysfunction in the immune system such as multiple sclerosis, psoriasis, diabetes (including latent autoimmune type 1 diabetes in adults (LADA)) and the like. The invention also provides analytical tools for diagnosing diseases and disorders that have an autoimmune origin. Another aspect of the present invention relates to pharmaceutical compositions comprising immunoglobulins of high activity, and methods for determining the activity levels of immunoglobulins in the pharmaceutical preparations. The present invention further provides a novel method for purification of a highly effective intravenous immunoglobulin (IVIG), wherein the resultant highly effective IVIG retains as much of its useful therapeutic characteristics in the donated bodily fluid that is the process input.

2. Related Art

Autoimmune and inflammatory diseases affect more than fifty million Americans. The immune system functions as the body's major defense against diseases caused by invading organisms. This complex system fights disease by killing invaders such as bacteria, viruses, parasites or cancerous cells while leaving the body's normal tissues unharmed. The immune system's ability to distinguish the body's normal tissues, or self, from foreign or cancerous tissue, or non-self, is an essential feature of normal immune system function. A second essential feature is memory, the ability to remember a particular foreign invader and to mount an enhanced defensive response when the previously encountered invader returns. The loss of recognition of a particular tissue as self and the subsequent immune response directed against that tissue produce serious illness.

Inflammation is involved in a large number of physiological and pathological conditions affecting animals and humans. Inflammatory responses can usually be traced to an immune response to an antigen, allergen, irritant, endotoxin or to tissue damage. The process is complex, involving a large number of components, many of which display pleiotropic effects, many of which are amplifiers or inhibitors of other components. While many instances of an inflammatory response are well controlled and self-limited, many pathologic conditions arise from uncontrolled or inappropriate responses, resulting in both acute and chronic conditions.

The immune system when operating normally is involved in precise functions such as recognition and memory of, specific response to, and clearance of, foreign substances (chemical and cellular antigens) that either penetrate the protective body barriers of skin and mucosal surfaces (transplanted tissue and microorganisms such as bacteria, viruses, parasites) or arise de novo (malignant transformation). The arsenal of the immune response is composed of two major types of lymphocytes that are either B-lymphocytes (B cells, responsible for producing antibodies which attack the invading microorganisms) or the T-lymphocytes (T cells, responsible for eliminating the infected or abnormal target cells) in cooperation with macrophages.

An autoimmune disease results from an inappropriate immune response directed against a self antigen (an autoantigen), which is a deviation from the normal state of self-tolerance. Self-tolerance arises when the production of T cells and B cells capable of reacting against autoantigens has been prevented by events that occur in the development of the immune system during early life. Several mechanisms are thought to be operative in the pathogenesis of autoimmune diseases, against a backdrop of genetic predisposition and environmental modulation. In general, antibodies (particularly, but not exclusively, IgG antibodies), acting as cytotoxic molecules or as a part of immune complexes, are the principal mediators of various autoimmune diseases, many of which can be debilitating or life-threatening.

The development and progression of certain forms of cancer and other diseases or disorders is similarly often associated with a pathogenic disturbance in the body's homeostasis. For example, certain forms of neoplastic diseases are associated with increased angiogenesis. In general, angiogenesis is a process of formation of new blood vessels in mammals and other animals. It is inherent to many activities of a normal human or animal body. Angiogenesis is vital for cellular growth and development, as well as wound-healing. Angiogenesis is also a necessary process for tumor growth.

Tumor progression is dependent on a number of sequential steps, including tumor-vascular interactions and recruitment of blood vessels. It is known that human and animal tumors produce a defined set of proangiogenic factors, which are typically offset by certain antiangiogenic factors produced in the normal mammalian body. When the proangiogenic and antiangiogenic activities are balanced, tumor mass cannot expand beyond a limited size, and the development of most mammalian cancers is arrested at a dormant mass of about 1-2 mm³ or smaller; cancers of this size often elude clinical detection and are cleared by the normal immune system of the mammal without any outward manifestation of the disease. However, due to a poorly understood molecular switch governed by various genetic and epigenetic factors, some tumours become excessively proangiogenic, which enables them to overproduce proangiogenic factors that overcome the antiangiogenic factors being produced by the normal mammalian body, thereby disturbing the homeostatic situation; in such cases, the tumors are able to recruit and sustain their own blood supply via the process of angiogenesis, resulting in the growth of the cancer into a palpable or otherwise clinically detectable tumor.

A vast number of pro- and anti-angiogenic factors have been described. Examples of proangiogenic factors include fibroblast growth factors, vascular endothelial growth factors, colony stimulating factors, interleukins, platelet-derived growth factors, angiopoietins, tumor-necrosis factors, matrix metalloproteinases (MMPs) and, in particular, transforming growth factor beta 1 (TGF-β1), intercellular adhesion molecules (ICAMs), hepatocyte growth factor, nerve growth factor, connective tissue growth factor, tenascin R, prolactin, growth hormone, placental lactogen, insulin-like growth factor 1, thymidine-phosphorylase, and the like. Examples of antiangiogenic factors include inteferons, tissue inhibitors of metalloproteinases (TIMPs), plasminogen, collagen, fibronectin, prolactin, growth hormones, thrombospondins, and fragments thereof. Among the most characterized antiangiogenic factors is angiostatin, a proteolytic fragment of plasminogen. As long as the expression, secretion or generation of pro- and antiangiogenic factors remains in equilibrium in the animal body, tumors will remain dormant. In certain diseases or disorders, however, this equilibrium in the activity of pro- and antiangiogenic factors is disrupted, which in turn can disturb the angiogenic balance resulting in the growth of new blood vessels, which can lead to angiogenesis-mediated pathologies.

Diagnosing and monitoring an activity of a disease or a disorder with autoimmune origin are both problematic in patients. Diagnosis is problematic because the spectrum of autoimmune diseases is often broad and ranges from subtle or vague symptoms to life threatening multi-organ failure. In addition, other diseases can be mistaken for autoimmune diseases, and vice versa. To further complicate a difficult diagnosis, symptoms of many autoimmune diseases may occur in combination with each other, and may continually evolve over the course of the disease. New symptoms in previously unaffected organs can develop over time. Testing of these highly variable diseases can therefore be complex, and is often misunderstood.

Monitoring disease activity is also problematic in caring for patients with malfunctions of the immune system. Some autoimmune diseases progress in a series of flares, or periods of acute illness, followed by remissions. In order to minimize devastating consequences of systemic organ damage often associated with autoimmune disorders, earlier and more accurate detection of disease flares would not only expedite appropriate treatment, but would reduce the frequency of unnecessary interventions. From an investigative standpoint, the ability to uniformly describe the activity of disease in individual organ systems or as a general measure is an invaluable research tool. Furthermore, a measure of disease activity can be used as a response variable in a therapeutic trial.

There is at present no cure for autoimmune diseases. However, there are a number of traditional approaches to treating autoimmune-related disorders and cancers that are known in the art. Among traditional treatments for patients with autoimmune diseases is an intravenous immunoglobulin (IVIG) therapy. Such therapy is typically accomplished by the intravenous administration to the patient of therapeutic preparations of normal polyspecific immunoglobulins, typically IgG immunoglobulins, obtained from pooled plasma or sera derived from up to thousands of healthy blood donors. Currently used commercially available preparations are made of intact IgG with a distribution of subclasses corresponding to that seen in normal serum and have a half-life of three weeks in vivo for IgG1, IgG2 and IgG4, and somewhat less for IgG3. Most of the preparations contain only traces of IgA, IgM and of Fc-dependent IgG aggregates. Owing to the large number of donors, the immunoglobulins used in IVIG therapy usually represent a wide spectrum of the expressed normal human IgG repertoire, including antibodies to external antigens, autoreactive antibodies and anti-antibodies (including anti-idiotypic antibodies). IVIG has been widely used for correction of immune deficiencies such as X-linked agammaglobulinemia, hypogamma-globulinemia, and acquired compromised immunity conditions, for treating various inflammatory and autoimmune diseases, and even cancer. U.S. Pat. No. 5,965,130 discloses the use of IVIG therapy for inhibition of tumor metastasis. However, the therapeutic effects of this treatment were disclosed in this patent to be short-lived, lasting between two weeks and three months, which thus does not provide long-term curative potential. Moreover, using these traditional approaches to achieve a long-term cure (even if that were possible) would likely be prohibitively expensive given the costs associated with researching, developing, manufacturing and obtaining regulatory approval for biological therapeutics such as IVIG. For at least these reasons, the use of IVIG in generally treating neoplastic diseases is not widespread.

The standard IVIG manufacturing process contains the following steps commonly used by most manufacturers: (a) Removal of Factor VIII and Factor IX using cryoprecipitation and ion exchange; (b) a series of cold alcohol processes (Cohn and Oncley cold ethanol process or variants including the Kistler & Nitschmann cold ethanol fractionation process) and absorption that results in a solution containing greater than 99% IgG; (c) a series of steps using low pH (<5.0), high temperature incubation (>30° C.) and harsh chemicals including solvents and detergents; (d) some manufacturers use a small amount of detergent (lubricant) and a filter that will remove any remaining viruses; (e) concentration by ultrafiltration to remove water; (f) a last sterile filtration to remove microbial contaminants; (g) adjust to proper pH (typically 4-6) and add stabilizers and fill; and (h) incubation at 30° C. for 2 weeks.

U.S. Pat. No. 6,932,969 discloses a method for preparing Ig fractions having reactivity to pathologic autoantibodies against actin, myosin, basic myelin protein, and tubulin. However, this method does not recognize a formation of pathologic autoantibodies against antiangiogenic factors and therefore it cannot be efficiently applied in the treatment of diseases with angiogenesis disorders.

WO 2008/006187 A2 discloses a method treatment of diseases with angiogenesis disorders having an autoimmune mechanism in their origin. In this method, a patient is administered a protein complex containing an angiogenic factor (or a portion thereof) and an immunomodulating moiety, which can either act as an immunostimulator or an immunosuppressor. Administration of the disclosed protein complex is described to result in a modulation of an immune response to the angiogenic factor in question. The main disadvantage of this method is the need of predefining an angiogenic factor which concentration exceed the normal level and for which there is an elevated levels of autoantibodies produced, and the need to identify (or even produce) a particular antibody, often a monoclonal antibody, that is specific for the predefined angiogenic factor—this need often raises the difficulty and the attendant costs of the procedure.

The primary goal in manufacturing IVIG for clinical use is to produce a safe product that retains as much of the useful therapeutic characteristics of the IgG in the donated plasma that is the process input. Safety focuses on the deactivation, destruction or removal of pathogens (such as virus) that may be present in donated plasma. As a positive result of this focus on pathogen elimination, currently available IVIG products are extremely safe. Safety also includes reducing or eliminating side effects. However, many of the manufacturing process steps used to damage virus also dramatically decrease the effectiveness of the IgG antibodies to the point where no long term clinical results can be achieved. Strong solvents, low pH, some detergents and high temperature incubation all reduce the efficacy of the IVIG product. Furthermore, virus filters can cause the accidental reduction or elimination of IgG antibodies that are required for effective lasting treatment success. Therefore, the negative result of the single focus on pathogen elimination is that the IgG in these products is generally ineffective at providing long term results.

Additionally, both the commercially used protocols, as well as the purification protocols disclosed in U.S. Pat. Nos. 6,069,236, 7,138,120, and 7,745,582 involve a number of steps that cause significant damage to the IVIG during the purification process. As a result, only a small fraction of the final purified IVIG product retains sufficient activity. However, a reliable method to assay the activity of IVIG at each step of purification is currently not available. Consequently, it is not possible to determine which steps lead to the most significant reduction in activity. This severely limits the scope of inventing new purification protocols which yield pure IVIG without a significant loss in activity.

Due to the loss of activity of IVIG associated with current isolation methods, the therapeutic effects of treatment with currently available purified IVIG are short-lived, lasting between two weeks and three months, which thus do not provide long-term curative potential. Moreover, currently no isolation method exists which allows the purification of a highly active IVIG, which is also free of active viral and microbial contaminants. For at least these reasons, the use of IVIG in generally treating cancer and autoimmune diseases is not widespread.

Despite claims over several decades of IVIG being suitable for treatment of cancer and auto-immune diseases, no long-term results have been documented. For the conditions and diseases that are treated with current preparations of IVIG, IVIG is merely satisfactory as a maintenance therapy. Furthermore, commercial IVIG preparations available today are produced using manufacturing processes that are almost entirely focused on destroying or disabling pathogenic viruses. As a positive result of this focus on virus elimination, IVIG products are very safe today. The negative result of the single focus is that the IgG in these products is ineffective at providing long-term curative potential.

Therapeutic apheresis is another method widely used for treatment of diseases mediated by antibodies circulating in patient's blood. One example of apheresis is plasmapheresis, a technique in which whole blood is withdrawn from a patient, anticoagulated, and separated into a plasma fraction and a corpuscular element fraction, generally by centrifugation or filtration. The purpose of therapeutic plasmapheresis is the removal from the patient's blood of pathologic plasma proteins or plasma proteins which are present in a noxiously high concentration, or, in cases of autoimmune diseases, specific antibodies or circulating antigen-antibody complexes. The chief drawback of this procedure is that only a limited volume of plasma can be drawn from a given donor, if no plasma replacement is given, which results in partial treatment. For more intensive treatments, the withdrawn plasma must be replaced either with purified albumin, or with normal plasma or other suitable plasma replacement fluid. This latter form of treatment is referred to as plasma exchange. Purified albumin is very expensive and does not provide all the proteins necessary for optimal replacement. Replacement with normal plasma is also expensive, and carries the risk of hepatitis. Moreover, the supply of normal plasma may soon be insufficient to fulfill the needs of all the patients who may benefit from such treatment. Additionally, while plasma exchange offers the quickest short-term answer to removing harmful autoantibodies, the production of autoantibodies by the immune system is not haulted, and the expensive procedure must be repeated on a regular basis.

Therefore there exists a need for an easy, inexpensive, safe, and efficient method of diagnosing and treating diseases having an autoimmune mechanism in their origin, including diseases with autoimmune angiogenesis disorders. Specifically, there exists a need for a diagnostic assay(s) that would not be limited to a specific autoimmune disease and would be suitable for assessing a general state of an immune system in a mammal. Additionally, there exists a need for a treatment method that will not necessitate subjecting a patient to recurrent procedures over the patient's life-time. The inventors have discovered how to make and use IVIG properly so that the treatment process of the present invention produces effective long-term results for most cancers and many other autoimmune conditions. The inventors also developed diagnostic assays that not only allow for an early and accurate diagnosis of immune abnormalities in a patient, but aid in monitoring the progression of the disease and recovery in response to treatments discussed herein. The treatment process of the present invention takes less than a week, requires low amounts of IVIG, has no significant side effects and lasts for many years in most patients. In addition, the inventors have developed analytical tools for measuring an activity of antibodies in the IVIG preparations, as well as for identifying patients that have weakened immune systems, indicative of being inflicted with disorders of autoimmune origin.

Therefore, the “highly effective” IVIG of the present invention is more potent as a therapeutic agent than the IVIG currently available. The highly effective IVIG of the present invention, synchronized with plasmapheresis of a patient, can therefore be used more effectively for the treatment of cancer and autoimmune diseases. Furthermore, the highly effective IVIG isolated by the methods of the present invention allows for the development of a treatment method that does not necessitate subjecting a patient to recurrent procedures over the patient's life-time.

### BRIEF SUMMARY OF THE INVENTION

The present invention is based on the unexpected discovery by the present inventors that cancer and various auto-immune diseases can be cured by detections and elimination of patient's aberrant immunoglobulin- (e.g., IgG-, IgM-, IgA-, IgE-, IgD-, etc.) mediated autoimmune responses and restoration of patient's immune system. It is further based on the unprecedented discovery by the present inventors that the development of cancer and various autoimmune disorders is intimately related to the pathogenic immunoglobulin-mediated autoimmune processes directed against organs, tissues, cells, molecules, and cellular processes in an animal, for example a mammal such as a human, and the discovery by the present inventors that substances capable of interfering with the activity of angiogenic factors can disturb the angiogenic balance, resulting in a new angiogenesis-mediated pathology. Specifically, the present inventors have unexpectedly discovered that there is an elevated concentration of autoantibodies, which may be IgG autoantibodies and which may be antibodies directed against one or more circulating signaling molecules, cellular receptors and angiogenesis factors and/or receptors normally found in the body, or which may be antibodies directed against anti-idiotypic antibodies, in the blood and tissues of cancer and autoimmune disease patients and experimental animals afflicted with these diseases. The presence of these antibodies in an early stage of a neoplastic disease suggests that there is a connection between a damaged adaptive immune system and the malignant growth, and supports the present inventors' discovery that a reversal of an autoimmune or idiotypic pathology can lead to inhibition of tumor and abnormal tissue growth and development. The present inventors therefore demonstrate herein that early detection of abeyant autoantibodies using analytical tools developed by the inventors and restoration of patient's immune system using certain methods of the present invention unexpectedly elicits a prolonged and often completely curative effect in a patient afflicted with a variety of diseases or disorders, such as cancers and other autoimmune disorders.

Thus, in a first aspect the present invention provides methods for diagnosing disease or disorders having autoimmune character in mammals, such as humans, mice, rats, dogs, cats, bovine species, porcine species, equine species, ovine species and the like. In some embodiments, a urine sample from a patient is assayed for presence of immunoglobulin light chains. In these embodiments, the amount of light chains in the urine sample is quantified, and a conclusion about a presence of an autoimmune disease or disorder is reached if the amount of light chains, secreted into urine during 24 hours exceeds at least about 30 mg.

In other embodiments, a general state of an immune system of a mammal is assessed based on an analysis of a patient's plasma sample. In these embodiments, plasma is analyzed for a ratio of immunoglobulin κ1 to κ2. In these embodiments, a sample of patient's plasma is subjected to an affinity purification, and amount of immunoglobulin is quantified in different elution peaks. In one embodiment, a patient is diagnosed with an autoimmune disorder if the amount of κ1 is less than about 0.05%×κ2.

Another aspect the present invention provides methods for treating and/or preventing diseases and disorders associated with a pathological autoimmune reaction in mammals, such as humans, mice, rats, dogs, cats, bovine species, porcine species, equine species, ovine species and the like. In one such embodiment, the invention provides methods of ameliorating, treating or preventing disease or disorder associated with the presence of one or more autoantibodies in the circulation of a mammal, comprising, in sequence: (a) DEPLETION of the concentration of pathogenic auto-antibodies and destructive proteins by removing a significant portion of these substances from the circulation of said mammal; and (b) ENRICHMENT of the patient's immune system with a complete set of antibodies including anti-idiotypic auto-antibodies by administering to said mammal one or more immunoglobulins in an amount sufficient to restore the immune system of said mammal to homeostasis.

According to certain aspects of the invention, the autoantibodies are advantageously removed from the circulation of the mammal by any method of removal of specific components from blood, most advantageously by apheresis methods such as plasmapheresis. In certain such embodiments, plasmapheresis is used over a period of from about one hour to about three hours to remove from about 100 ml to about 1000 ml, and typically from about 600 ml to about 800 ml, of plasma from the mammal, thereby removing much of the cohort of toxic autoantibodies from the mammal since such autoantibodies are found in the plasma.

Following apheresis, e.g., plasmapheresis, the immune system of the mammal is restored to homeostasis or baseline status by an infusion of immunoglobulins, preferably mixed gamma globulins or IgG, into the mammal, preferably via an intravenous route (IVIG). In certain other embodiments of the invention, the immune system of the mammal is restored to homeostasis or baseline status by an infusion of immunoglobulins without first subjecting the mammal to apheresis, e.g., plasmapheresis. In some embodiments, the IVIG preparations used in this aspect of the invention have at least 20% active immunoglobulins, as determined by assays disclosed herein. In other embodiments, the IVIG preparations have at least 30% active immunoglobulins. In yet other embodiments, the IVIG preparations used in this aspect of the invention have at least 45% active immunoglobulins. The IVIG preparations used in the invention can also have more than 50% active immunoglobulins.

The immunoglobulins are preferably administered to the mammal in fixed doses over a period of from about one day to about ten days, preferably from about one day to about eight days, from about one to five days, and more preferably in about one day, two days, three days, four days, five days, six days, seven days, eight days, nine days or ten days. In certain such embodiments, the immunoglobulins are administered to the mammal in an amount totaling from about 2.5 grams to about 200 grams, from about 5 grams to about 100 grams, from about 5 grams to about 80 grams, from about 5 grams to about 40 grams, from about 5 grams to about 30 grams, from about 5 grams to about 25 grams, from about 5 grams to about 20 grams, from about 5 grams to about 15 grams, from about 5 grams to about 10 grams, and advantageously about 10 grams. The immunoglobulins are advantageously administered to the mammal according to a fixed schedule, depending on the number of cycles or days over which immunoglobulins are administered to the patient. For example, in a 5-cycle administration schedule, immunoglobulins may be administered as follows: (a) on Day 2, 0 to 2 grams (e.g., 1.25 grams); (b) on Day 3, 0 to 4 grams (e.g., 2.5 grams); (c) on Day 4, 0 to 5 grams (e.g., 0 grams); (d) on Day 5, 0 to 7 grams (e.g., 5 grams); and (e) on Day 6, 0 to 10 grams (e.g., 10 grams). Adjustments to the schedule may be made as necessary to achieve the total amount of immunoglobulin as outlined above, administered over a total of one day, two days, three days, four days, five days, six days, seven days, eight days, nine days or ten days. Optimally, as few a number of days or cycles of immunoglobulin administration as possible is used to provide maximal benefit (in terms of effectiveness, safety and comfort) to the patient.

Immunoglobulin is a complex medication made from donator plasma that contains hundreds of millions of different antibodies and some trace proteins. Immunoglobulin, as a term used in this application, also refers to substitutes for immunoglobulin. Substitutes may include medications that include immunoglobulin (for example whole blood and plasma) or may be subsets of the antibodies and proteins found in immunoglobulin including synthesized antibodies and other synthetic molecules which mimic the functionality of components of immunoglobulin.

Immunoglobulin varies widely in composition, concentration and activity level. The most effective immunoglobulin will be sourced from younger donors who have healthy immune systems. Excessive processing of donor immunoglobulin can damage critical components during manufacturing. This damage can render a manufacturer's immunoglobulin product partially or totally ineffective. This damage can and should be assessed prior to use. Even after initial assessment, a seemingly minor change in manufacturing process can change the effectiveness for this treatment process.

The methods of the invention are advantageously used in treatment, amelioration and/or prevention of a variety of diseases and disorders, including but not limited to a neoplastic disease, an autoimmune disease or disorder, a cardiovascular disease, a respiratory disease, a urinary tract disease, a gastrointestinal tract disease, a reproductive disorder, a nervous system disease, a mental disorder, a musculoskeletal system disease, an endocrine disease, a connective tissue disease, a skin disease, a transplantation disease, a disease related to one or more sensory organs, and an infectious disease. Most preferably, the methods of the invention are used to treat or prevent neoplastic diseases (including but not limited to carcinomas, sarcomas, lymphomas, leukemias, germ cell tumors, blastomas and the like, and particularly non-brain carcinomas or sarcomas), or autoimmune diseases or disorders (including but not limited to Lupus erythematosus, Addison's disease, Alopecia areata, Alzheimer disease, Ankylosing spondylitis, Atherosclerosis, Antiphospholipid antibody syndrome, Autoimmune hepatitis, Autoimmune inner ear disease, Bullous pemphigoid, Behçet's disease, Cardiac infarction, Coeliac disease, Chagas disease, Chronic obstructive pulmonary disease, Crohns Disease, Cellulitis, Dermatomyositis, Dilated cardiomyopathy, graft-versus-host disease (GVHD), host-versus graft disease (HVGD), Endometriosis, Epilepsy, Goodpasture's syndrome, Graves' disease, Guillain-Barré syndrome, Hidradenitis suppurativa, IgA nephropathy, Kawasaki disease, Interstitial cystitis, Idiopathic thrombocytopenic purpura, Morphea, Multiple sclerosis, Pathologic obesity, Pernicious anaemia, Schizophrenia, Psoriasis, Sjögren's syndrome, Scleroderma, Rheumatoid arthritis, Dermatomyositis, Diabetes mellitus type 1 (which may be latent autoimmune diabetes in adults or LADA), Hashimoto's thyroiditis, Addison's disease, Pemphigus vulgaris, Autoimmune haemolytic anaemia, Vasculitis, Vitiligo and Wegener's granulomatosis.

In certain applications of the present invention, it may be desirable to administer at least one anticoagulant to the patient, such as glucose sodium citrate, heparin, ximelagatran, argatroban, lepirudin, bivalirudin, warfarin, phenindione, acenocoumarol and phenprocoumon. In additional aspects of the invention, it is desirable to administer to the patient, immediately prior to, during or immediately following administration of the immunoglobulins to the patient, at least one antihistamine (including but not limited to diphenhydramine, loratadine, desloratadine, fexofenadine, meclizine, pheniramine, cetirazine, promethazine, chlorpheniramine, levocetirazine, cimetidine, famotidine, ranitidine, ciproxifan and clobenpropit) or at least one non-steroidal antiinflammatory agent (including but not limited to aspirin, ibuprofen, naproxen, diclofenac, aceclofenac and licofelone). In preferred such aspects, the patient is administered diphenhydramine immediately prior to being infused with immunoglobulins.

The present relates to a method of purifying a IVIG preparation, free of active viral and microbial contaminants, that is highly effective as a therapeutic agent for treating diseases or disorders in a mammal.

Thus, in one embodiment the invention provides a method of purifying a human IVIG from a bodily fluid, wherein the resultant IVIG is suitable for therapeutic use, the method comprising the steps of:
(a) removing one or more components of coagulation pathway from the bodily fluid;
(b) adding one or more alcohols to the bodily fluid to remove undesired proteins;
(c) concentrating the bodily fluid under conditions that avoid activation of the complement pathway in the bodily fluid;
(d) treating the bodily fluid to eliminate one or more active viral and microbial contaminants; and
(e) assaying the activity of the IVIG at least after (d) to obtain a purified IVIG from the plasma protein concentrate, wherein the purified IVIG is a highly effective IVIG for treating one or more disease or disorder in a mammal.

Other preferred embodiments of the present invention will be apparent to one of ordinary skill in light of what is known in the art, in light of the following drawings and description of the invention, and in light of the claims.

### BRIEF DESCRIPTION OF THE DRAWINGS

FIG. 1 illustrates an affinity chromatography diagram for analyzing a plasma sample of the healthy person. Immunoglobulins κ1 to κ2 is 2.5%.

FIG. 2 illustrates an affinity chromatography diagram for analyzing a plasma sample of the cancer patient. Immunoglobulins κ1 to κ2 is 0.04%.

### DETAILED DESCRIPTION OF THE INVENTION

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention.

The embodiment(s) described, and references in the specification to “one embodiment”, “an embodiment”, “an example embodiment”, etc., indicate that the embodiment(s) described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

### Definitions

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are described hereinafter.

As used herein, the term “immune response” is meant to refer to a process of a detection and reaction of an organism to an agent. “Humoral Immune Response” (or HIR) describes the aspect of immunity that is mediated by secreted antibodies (as opposed to cell-mediated immunity, which involves T lymphocytes) produced in the cells of the B lymphocyte lineage (B cells). B Cells (with co-stimulation) transform into plasma cells which secrete antibodies. The co-stimulation of the B cell can come from another antigen presenting cell, like a dendritic cell. Humoral immunity is so named because it involves substances found in the humours, or body fluids.

A term “immunological intolerance,” as used herein, is referred to a process of developing an immune response to a self antigen. Immunological intolerance develops as a result of a failure of an organism to recognize its own constituent parts as self, which allows an immune response against them. Consequently, a term “immunological tolerance” refers to a lack of immune response to the antigen. The immunological tolerance can be restored by manipulating the immune system of an organism.

Terms “abnormal angiogenesis,” “altered angiogenesis,” or “angiogenic disbalance” are used interchangeably, and refer to a process of formation of new blood vessels that has taken on a pathological character, not ordinarily found in healthy organisms. Consequently, the term “angiogenic balance” refers to a process of blood vessel formation that occurs in the normally-functioning organ.

As used herein, the phrases “pre-clinical stage” of “pre-clinical phase” of a disease refer to a period at which the disease is early in its natural history and before the onset of any symptoms. The phrases “clinical stage” or “clinical phase” of a disease are meant to refer to a period during which symptoms characteristic of a certain disease have developed. Depending on the severity of the symptoms and the biological age of the disease, clinical phase can be divided into an early phase and a late phase.

“Patients” contemplated for application of the invention methods described herein are mammals including humans, domesticated animals, and primates (e.g. a marmoset or monkey). The patient may be human or a non-human animal. As used herein, the term “tumor” refers to a malignant tissue comprising transformed cells that grow uncontrollably.

As used herein, an animal (e.g., a mammal) that is “predisposed to” a disease or disorder is defined as an animal that does not exhibit a plurality of overt physical symptoms of the disease or disorder but that is genetically, physiologically or otherwise at risk for developing the disorder. In the present invention, the identification of an animal (such as a mammal, including a human) that is predisposed to, at risk for, or suffering from a given physical disease or disorder may be accomplished according to the diagnostic methods of the present invention described in detail herein, and may be confirmed using standard art-known methods that will be familiar to the ordinarily skilled clinician, including, for example, radiological assays, biochemical assays (e.g., assays of the relative levels of particular peptides, proteins, electrolytes, etc., in a sample obtained from an animal), surgical methods, genetic screening, family history, physical palpation, pathological or histological tests (e.g., microscopic evaluation of tissue or bodily fluid samples or smears, immunological assays, etc.), testing of bodily fluids (e.g., blood, serum, plasma, cerebrospinal fluid, urine, saliva, semen and the like), imaging, (e.g., radiologic, fluorescent, optical, resonant (e.g., using nuclear magnetic resonance (“NMR”) or electron spin resonance (“ESR”)), etc. Once an animal has been identified as suffering from or predisposed to a disease or disorder by one or more such methods, the animal may be aggressively and/or proactively treated to prevent, suppress, delay or cure the disease or disorder, for example using the treatment methods of the present invention described in detail herein.

As used herein when referring to any numerical value, the term “about” means a value of ±10% of the stated value (e.g., “about 50° C.” encompasses a range of temperatures from 45° C. to 55° C., inclusive; similarly, “about 100 grams” encompasses a range of masses from 90 grams to 110 grams, inclusive).

As used herein, the term “immunoglobulin” means an antibody or fragment (e.g., Fab, Fab′2, Fc, etc.) thereof, or a preparation of immunoglobulins that can be prepared according to art-known methods or that are commercially available. Immunoglobulins used in accordance with the present invention may of any class, subclass and isotype, including IgG, IgM, IgA, IgD and IgE; preferably, IgG immunoglobulins are used in the methods of the present invention.

As used herein, the term “intravenous immunoglobulin” or “IVIG” is a blood product administered intravenously. It contains the pooled IgG extracted from the plasma of over one thousand blood donors. IVIG is given as a plasma protein replacement therapy (IgG) for immune deficient patients who have decreased or abolished antibody production capabilities. In these immune deficient patients, IVIG is administered to maintain adequate antibodies levels to prevent infections and confers a passive immunity. The precise mechanism by which IVIG suppresses harmful inflammation has not been definitively established but is believed to involve the inhibitory Fc receptor. However, the actual primary target(s) of IVIG in autoimmune disease are unclear. IVIG may work via a multi-step model where the injected IVIG first forms a type of immune complex in the patient. Once these immune complexes are formed, they interact with activating Fc receptors on dendritic cells which then mediate anti-inflammatory effects helping to reduce the severity of the autoimmune disease or inflammatory state. Additionally, the donor antibody may bind directly with the abnormal host antibody, stimulating its removal. Alternatively, the massive quantity of antibody may stimulate the host's complement system, leading to enhanced removal of all antibodies, including the harmful ones. IVIG also blocks the antibody receptors on immune cells (macrophages), leading to decreased damage by these cells, or regulation of macrophage phagocytosis. IVIG may also regulate the immune response by reacting with a number of membrane receptors on T cells, B cells, and monocytes that are pertinent to autoreactivity and induction of tolerance to self.

As used herein, the term “highly effective IVIG” refers to an IVIG preparation isolated from a bodily fluid via a purification process, wherein the final purified IVIG retains as much of the activity and/or useful therapeutic characteristics of the IgG in the donated bodily fluid that is the process input. In some embodiments, the purified IVIG retains at least about 25% or at least about 30% of the activity and/or useful therapeutic characteristics of the IgG in the donated bodily fluid. In a preferred embodiment, the purified IVIG retains greater than about 50% of the activity and/or useful therapeutic characteristics of the IgG in the donated bodily fluid.

As used herein, the term “coagulation pathway” refers to the complex cascade of processes by which blood forms clots. Coagulation is an important part of hemostasis (the cessation of blood loss from a damaged vessel), wherein a damaged blood vessel wall is covered by a platelet and fibrin-containing clot to stop bleeding and begin repair of the damaged vessel. Disorders of coagulation can lead to an increased risk of bleeding (hemorrhage) or obstructive clotting (thrombosis). The coagulation cascade of secondary hemostasis has two pathways which lead to fibrin formation. These are the contact activation pathway (formerly known as the intrinsic pathway), and the tissue factor pathway (formerly known as the extrinsic pathway). It was previously thought that the coagulation cascade consisted of two pathways of equal importance joined to a common pathway. It is now known that the primary pathway for the initiation of blood coagulation is the tissue factor pathway. The pathways are a series of reactions, in which a zymogen (inactive enzyme precursor) of a serine protease and its glycoprotein co-factor are activated to become active components that then catalyze the next reaction in the cascade, ultimately resulting in cross-linked fibrin. Coagulation factors are generally indicated by Roman numerals, with a lowercase a appended to indicate an active form. The coagulation factors are generally serine proteases. There are some exceptions. For example, FVIII and FV are glycoproteins, and Factor XIII is a transglutaminase. Serine proteases act by cleaving other proteins at specific sites. The coagulation factors circulate as inactive zymogens. The coagulation cascade is classically divided into three pathways. The tissue factor and contact activation pathways both activate the final common pathway of factor X, thrombin and fibrin.

As used herein, the term “complement system” is a biochemical cascade that helps, or “complements” the ability of antibodies to clear pathogens from an organism. It is part of the immune system called the innate immune system that is not adaptable and does not change over the course of an individual's lifetime. However, it can be recruited and brought into action by the adaptive immune system. The complement system consists of a number of small proteins found in the blood, generally synthesized by the liver, and normally circulating as inactive precursors (pro-proteins). When stimulated by one of several triggers, proteases in the system cleave specific proteins to release cytokines and initiate an amplifying cascade of further cleavages. The end-result of this activation cascade is massive amplification of the response and activation of the cell-killing membrane attack complex. Over 25 proteins and protein fragments make up the complement system, including serum proteins, serosal proteins, and cell membrane receptors. They account for about 5% of the globulin fraction of blood serum.

As used herein, the term “apheresis” is a medical technology in which the blood of a donor or patient is passed through an apparatus that separates out one particular constituent and returns the remainder to the circulation. It is thus an extracorporeal therapy i.e., a medical procedure which is performed outside the body. Depending on the substance that is being removed, different processes are employed in apheresis. For example, if separation by density is required, centrifugation is the most common method. Other methods involve absorption onto beads coated with an absorbent material and filtration. There are numerous types of apheresis which include plasmapheresis, erythrocytapheresis, plateletpheresis, leukapheresis, etc.

As used herein, the term “plasmapheresis” involves the removal, treatment, and return of blood plasma or components of blood plasma from blood circulation. It is thus an extracorporeal therapy i.e., a medical procedure which is performed outside the body. The method can also be used to collect plasma for further manufacturing into a variety of medications. Three procedures are commonly used to separate the plasma from the blood cells: (1) discontinuous flow centrifugation; (2) continuous flow centrifugation; and (3) plasma filtration. After plasma separation, the blood cells are returned to the person undergoing treatment, while the plasma, which contains the antibodies, is first treated and then returned to the patient in traditional plasmapheresis. An important use of plasmapheresis is in the therapy of autoimmune disorders, where the rapid removal of disease-causing autoantibodies from the circulation is required in addition to other medical therapy.

By “bodily fluid” is intended any fluid sample obtained from a subject, including but not limited to plasma, blood, serum, cerebrospinal fluid, synovial fluid, colostrum, and nipple aspirates. Bodily fluid may be obtained using any methodology known in the art.

Other terms used in the fields of medicine, pharmacology and immunology as used herein will be generally understood by one of ordinary skill in the applicable arts.

### Overview

It is an object of the present invention to provide methods of diagnosing and treating autoimmune-related diseases and disorders in mammals. In some aspects, immunological health of a mammal will be assessed for presence of any weakening of immune system prior to treatment. In other aspects, it is not necessary to identify a dysfunction in the immune system of a mammal before correction of that pathogenic immune response with the methods of the present invention. In certain aspects, the methods of the invention comprise altering autoimmune processes by production of immunological tolerance of organs, tissues, cells, molecules, or cellular processes and factors. In certain other aspects, the methods of the invention comprise altering autoimmune processes by production of immunological tolerance of antiangiogenic factors. Yet in other aspects, the methods of the invention comprise altering autoimmune processes by providing certain anti-idiotypic auto-antibodies that would normally remove the pathogenic auto-antibodies causing the autoimmune-related diseases and disorders in mammals.

The highly effective IVIG of the present invention includes immunoglobulins that may be of any class, subclass and isotype, including but not limited to IgG, IgM, IgA, IgD and IgE, or mixtures thereof, but preferably are enriched in (i.e., predominately contain) IgG immunoglobulins. Also contemplated for use herein are aqueous solutions containing higher concentrations of IVIG, such as those containing approximately 25%-75% w/v or w/w IVIG. In one embodiment, the highly effective IVIG of the present invention is substantially pure. In some embodiments, the highly effective IVIG contains greater than about 50% w/v or w/w, preferably greater than 75% w/v or w/w, and more preferably greater than about 90% w/v or w/w, of IgG immunoglobulins in the preparation.

### Diagnostic Assays

Another aspect of the present invention is directed to methods of assessing a state of an immune system in a mammal. In some embodiments, the present invention provides methods for diagnosing an autoimmune disorder in a patient. For the purposes of the present invention, the terms “diagnosis” or “diagnosing” shall mean making a determination that a patient is afflicted with an autoimmune disease or disorder with at least 90%, preferably 95%, more preferably 99% accuracy. In other words, no more than 10 out 100, preferably 5 out of 100, and even more preferably I out of 100 patients diagnosed with an autoimmune abnormality using methods described herein will be considered falsely diagnosed. In other embodiments, diagnosis of an autoimmune disease or disorder made with methods of the present invention will have an adequate accuracy required for an approval of such methods by the US Food and Drug Administration.

In certain embodiments, a method of diagnosing an autoimmune disease or disorder in a mammal comprises assessing a urine sample from the mammal for a presence of light chains immunoglobulins. In some embodiments, the presence of light chain immunoglobulins in the urine sample can be conducted using affinity chromatography. In some embodiments, protein affinity chromatography will be used. “Protein affinity chromatography” refers to the separation or purification of substances and/or particles using a particular protein, where the particular protein is generally immobilized on a solid phase. By “solid phase” is meant a non-aqueous matrix to which the protein can adhere or be covalently bound. The solid phase can comprise a glass, silica, polystyrene, or agarose surface for immobilizing the protein, for instance. The solid phase can be a purification column, discontinuous phase of discrete particles, packed bed column, expanded bed column, membrane, etc. In certain embodiments the protein suitable for use in the methods of the present invention is selected from the group consisting of protein L, protein A, protein G, or a combination thereof. When used herein, the term “protein A”, “protein L”, or “protein G” encompass proteins A, L, or G recovered from a native source thereof, and proteins A, L or G produced synthetically (e.g. by peptide synthesis or by recombinant techniques), including variants or derivatives thereof which retain the ability to bind light chain immunoglobulins. In one embodiments, the urine sample is analyzed using protein L affinity chromatography.

Light chain immunoglobulins present in the urine sample can be reversibly bound to, or adsorbed by, the protein L-Sepharose. Examples of protein L affinity sorbents for use in protein L affinity chromatography herein include, but are not limited to, sorbents manufactured by Sigma-Aldrich or Thermo Fisher Scientific Inc. In certain embodiment, the solid phase for the protein L affinity chromatography can be equilibrated with a suitable buffer before chromatographic separation of the urine sample. A skilled artisan will be familiar with an abundance of equilibration buffers available for use in affinity chromatography. A choice of the equilibration buffer can also depend on the manufacturing protocol for the specific affinity column. For example, the equilibration buffer can be 20 mM Na₂HPO₄, 0.15 M NaCl, pH 8.0 In some embodiments, a urine sample can be loaded directly onto the equilibrated protein L column. In other embodiments, the urine sample can be diluted to an artisan's preference with a loading buffer. The sample can then be loaded on the equilibrated solid phase using a loading buffer, which can be the same as the equilibration buffer. The amount of sample loaded on the column will depend on a number of factors, such as an availability of the sample and column's capacity. In some embodiments, at least about 100 ml of the sample is loaded on the column. In other embodiments, at least about 200 ml of the sample is loaded.

After the entire urine sample is loaded onto the column, the column can be washed with at least 2 column volumes with a wash buffer. In some embodiments, the column will be washed with about at least 3-5 column volumes of the wash buffer. Suitable buffers for this purpose include, but are not limited to, Tris, phosphate, MES, citrate, MOPSO buffers, and combinations thereof.

The preferred pH of the wash buffer is at least about 7. In some embodiments, the pH of the wash buffer is about 6. After the completing of the wash, light chain immunoglobulins can be recovered from the protein L column using an elution buffer. The protein may, for example, be eluted from the column using about 1-2 column volumes of elution buffer having a low pH, e.g. in the range from about 2 to about 4, and preferably in the range from about 2.3 to about 3.5. Examples of elution buffers for this purpose include citrate or glycine-HCl buffers. In some embodiments, the pH of the elution buffer will be about 3.5. In one embodiment, the pH of the elution buffer is about 2.3, in one embodiment, the light chain IgG's are recovered from the protein L column using a two-step process, wherein the light chain IgG's elute in two separate batches. In one aspect, the first batch of light chain IgG's are eluted at a pH of about 5 and the second batch of light chain IgG's are eluted at a pH of about 3. In one aspect, the light chain IgG's eluted at a pH of about 5 are the bound IgG κ1. In one aspect, the light chain IgG's eluted at a pH of about 3 are the bound IgG κ2. In one embodiment, the light chain IgG's eluting at a pH of about 5 are the IgG's that are relevant to the present invention.

In certain embodiments, the total amount of light chain IgG's eluted from the protein L column will be determined. Any method for determination of protein concentration can be used for the purposes of quantifying the amount of immunoglobulins light chain recovered from the affinity column. One such method uses a well-known measurement of protein absorbance at 280 nm. In one aspect, the amount of light chain IgG's in the urine sample are normalized. In one embodiment, the chain IgG's in the urine sample are normalized with respect to the creatine present in the urine sample. In one embodiment, the amount of creatinine in a urine sample is determined by a creatinine clearance test. Creatinine clearance tests measure the level of creatinine in a subject's blood and urine. Creatine is formed when food is changed into energy through metabolism. Creatine is broken down into creatinine, which is taken out of the blood by the kidneys and then passed out of the body in urine.http://www.webmd.com/hw-popup/kidneys

Once the amount of light chain protein in the urine sample is determined, a diagnosis of an autoimmune disease or disorder can be made. In some embodiments, presence of at least about 1 mg of immunoglobulin light chain in about 100 ml (or about 30 mg in total urine, collected during 24 hours) of starting urine sample will indicate a presence of autoimmune abnormality. In one embodiment, the urine sample is the first urine collected in the morning.

In other embodiments, a method of diagnosing an autoimmune disease or disorder in a mammal comprises assessing a plasma sample from the mammal for a presence of immunoglobulin κ. In some embodiments, the presence of immunoglobulin κ in the plasma sample can be conducted using an affinity chromatography. In some embodiments, protein affinity chromatography will be used. In certain embodiments the protein suitable for use in the methods of the present invention is selected from the group consisting of protein L, protein A, protein G, or a combination thereof. In one embodiments, the plasma sample is analyzed using protein A affinity chromatography. Examples of protein A affinity chromatography columns for use in protein A affinity chromatography herein include protein A immobilized onto a controlled pore glass backbone, including the PROSEP-A™ and PROSEP-vA™ columns (Millipore Inc.); protein A immobilized on a polystyrene solid phase, e.g. the POROS 50A™ column (Applied BioSystems Inc.); or protein A immobilized on an agarose solid phase, for instance the rPROTEIN A SEPHAROSE FAST FLOW™ or MABSELECT™ columns (Amersham Biosciences Inc.).

Affinity chromatography for analyzing a plasma sample will be conducted according specifically designed protocol. The solid phase for the protein A affinity chromatography can be equilibrated with a suitable buffer before chromatographic separation of the plasma sample. In some embodiments, a plasma sample can be loaded directly onto the equilibrated protein A column. In other embodiments, the plasma sample can be diluted with a loading buffer. The sample can then be loaded on the equilibrated solid phase using a loading buffer, which may be the same as the equilibration buffer. The amount of sample loaded on the column will depend on a number of factors, such as an availability of the sample and column's capacity. In some embodiments, at least about 1 ml of the sample is loaded on the column. In other embodiments, at least about 0.2 ml of the sample is loaded.

After the entire plasma sample is loaded onto the column, the column can be washed with at least 1 column volumes with a wash buffer. In some embodiments, the column will be washed with at least about 10-15 column volumes of the wash buffer. The preferred pH of the wash buffer is about 7. After washing the column elution of absorbed immunoglobulins are eluted by step-decreasing of pH of eluting buffer. Certain immunoglobulins (termed herein as “immunoglobulins κ1”) will elute at pH<6, preferably at pH 5. Immunoglobulins κ1 will be collected and quantified using methods generally available to a person of skill in the art and described herein. Certain other immunoglobulins (termed herein as “immunoglobulins κ2”) will not elute at pH 5, and will remain bound to the column. These immunoglobulins can be recovered from the protein A column using about 1-2 column volumes of elution buffer having a low pH, e.g. in the range from about 2 to about 4, and preferably in the range from about 2.3 to about 3.5. In some embodiments, the pH of the elution buffer will be about 3.5. In one embodiment, the pH of the elution buffer is about 2.3.

Once immunoglobulin κ2 fraction is collected from the column, the amount of immunoglobulin κ2 can be quantified using methods described herein and generally known to a person of ordinary skill in the art. In some embodiments, the amount of immunoglobulin κ2 is compared to the amount of immunoglobulin κ1. In certain embodiments, an autoimmune disorder is diagnosed if the amount of immunoglobulin κ1 is less that at least about 0.1%×the amount of immunoglobulin κ2. In other embodiments, an autoimmune disorder is diagnosed if the amount of immunoglobulin κ1 is less than at least about 0.05%×the amount of immunoglobulin κ2. A healthy patient sample will comprise at least approximately 0.05% κ1 fraction of the κ2 fraction.

### Process of Immune System Restoration

Another aspect of the present invention is directed to a process of restoring an immune system of a patient in need thereof (one embodiment of such a process is referred to herein by its commercial name, the Eiger Immune Restoration Process or EIRP (Eiger Health Partners LLP; Amagansett, N.Y.). In some embodiments, the process of the present invention comprises restoration of immunological tolerance of organs, tissues, cells, molecules, or cellular processes and factors in a patient in need thereof. An immunological intolerance referred to herein is not limited to an intolerance of a specific organ, tissue, cell, molecule, cellular process or factor, and encompasses normally functioning as well as diseased, disordered, or otherwise compromised organs, tissues, cells, molecules, or cellular processes and factors.

In one embodiment, the process of the present invention comprises restoration of immunological tolerance of and non-interference with normal angiogenesis factors and pathways. An angiogenic factor referred to herein includes, but is not limited to, any naturally occurring substance capable of participating in an angiogenic process of an organism. Such factor can be proangiogenic, or capable of promoting the process of angiogenesis, or antiangiogenic, or capable of inhibiting angiogenesis. Examples of the proangiogenic factors include, but are not limited to, fibroblast growth factors, vascular endothelial growth factors, colony stimulating factors, interleukins, platelet-derived growth factors, angiopoietins, tumor-necrosis factors, matrix metalloproteinases and, in particular, transforming growth factor beta 1, intercellular adhesion molecule, hepatocyte growth factor, nerve growth factor, connective tissue growth factor tenascin-R, prolactin, growth hormone, placental lactogen, insulin-like growth factor 1, thymidine-phosphorylase. Examples of the antiangiogenic factors include, but are not limited to, inteferons, tissue inhibitors of metalloproteinases, fibroblast growth factors, placental endothelial growth factors, vascular endothelial growth factors, plasminogen, collagen, fibronectin, prolactin, growth hormones, placental lactogens, thrombospondins and fragments thereof. In some embodiments, the present invention is directed to a process of restoring an immunological tolerance of an antiangiogenic factor in a mammal. In one embodiment, the poorly tolerated antiangiogenic factor is angiostatin, which is a proteolytic fragment of plasminogen. Therefore, one embodiment of the present invention relates to a process of restoring an immunological tolerance of angiostatin in a mammal.

In another embodiment, the process of immune system restoration of the present invention comprises altering autoimmune processes by providing certain anti-idiotypic auto-antibodies that would normally remove the pathogenic auto-antibodies causing the autoimmune-related diseases and disorders in mammals. This aspect of the invention is based on the discovery by the present inventors that in certain disease states, such as certain autoimmune diseases or disorders that may or may not involve altered angiogenesis, there is a notable decrease or absence in the amount of anti-idiotypic autoantibodies, that would normally remove pathogenic auto-antibodies causing the disease state, in the circulation and tissues of patients. The methods of the present invention, as outlined in detail below and as exemplified by the EIRP, can be used to restore the levels of anti-idiotypic antibodies in such patients which may in itself be sufficient to eradicate or at least control the autoimmune disease or disorder, including neoplastic diseases, by providing circulating anti-idiotypic antibodies that can bind to and eliminate pathogenic autoantibodies.

The methods of the present invention, e.g., the EIRP, can be performed at any time during the period manifested by an abnormal immune response. In one embodiment of the present invention, the immune system is restored at the pre-clinical stage of a disease characterized by an abnormal immune response. At this stage, the immune system restoration has a preventative effect, in that it inhibits a development of any symptoms associated with the disease and halts its progression into a clinical phase. In another embodiment, the immune system is restored at a clinical stage of a disease. Restoration of the immune system at the clinical phase has a treatment effect, in that it eliminates pathologic symptoms and completely cures the disease.

In some embodiments, a process of the invention for restoring an immune system in a mammal comprises two phases. In one embodiment, Phase 1 comprises detoxifying the blood of said mammal by removing autoantibodies. In these embodiments, Phase 1 is followed by Phase 2, which comprises administering to the mammal a preparation of immunoglobulins in an amount sufficient to modulate an immune response to the autoantibodies and to B-cells that produce the pathogenic autoantibodies.

It is understood that the description contained herein is but one exemplary embodiment for removing pathogenic autoantibodies from a patient's circulation. In some embodiments, autoantibodies are removed by apheresis, for example by plasmapheresis. In certain embodiments, plasmapheresis will remove between about 15% about 30% of the patient's total circulating plasma. A skilled artisan will be familiar with typical procedures used to perform apheresis techniques such as plasmapheresis. In some embodiments, plasmapheresis can be performed by a discontinuous flow centrifugation. These embodiments requires one venous catheter. Blood is removed in batches of about 100 to about 700 ml at a time and centrifuged to separate plasma from blood cells. In one embodiment, 600 ml of blood is removed over a period of about 0.5 to about 2 hours. In another embodiment, 600 ml of blood is removed in a period of about 1 to about 1.5 hours. In other embodiments, apheresis can be performed by a continuous flow centrifugation. These embodiments entail use of two venous lines. Blood can be removed in about 50 to about 300 ml batches at a time while plasma is spun out continuously. In yet other embodiments, plasma can be removed by a process of plasma filtrations. In these embodiments, the plasma can be filtered using standard hemodialysis equipment. These embodiments often require use of two venous lines, wherein blood is continuously removed in about 20 to about 100 ml batches. After plasma is separated using any of the methods described herein, the blood cells are returned to the person undergoing treatment.

In some embodiments, the plasma, which contains pathogenic autoantibodies, can be treated to remove pathogenic antibodies and returned into the patient's circulation. In one such embodiment, the pathogenic antibodies can be removed by cryo-precipitation. In this embodiment, heparin is added to removed plasma and the plasma is frozen (at about 0° C. to about −20° C. for several hours and subsequently thawed. After thawing of the plasma, precipitated protein is removed by centrifugation, and the remaining plasma is returned into the patients circulation. In another embodiment, the pathogenic antibodies can be removed by passing the plasma over a solid-phase matrix (e.g., in a column) having an affinity for autoantibodies (or antibodies in general). Such methods of affinity chromatography for removing specific antibodies or classes of antibodies include the use of Protein A affinity matrices, Protein G affinity matrices, antibody-specific affinity matrices (which may use, for example, antibodies or fragments thereof immobilized on the solid phase that will bind the pathogenic antibodies in the plasma as it is placed into contact with the solid phase affinity matrix). Other such affinity-based methods of removing pathogenic autoantibodies will be familiar to those of ordinary skill in the art. In other embodiments, a targeted percent of circulating antibodies of a chosen type (e.g., IgG antibodies), whether normal or pathogenic, can be removed using special absorption filters. An example of such filter is, but is not limited to, an FcRn column, which is available commercially from multiple manufacturers that will be familiar to those of ordinary skill in the art. In yet another embodiment, the removed plasma can be treated with a medication capable of destroying IgG-producing B-cells. An example of such medication is, but is not limited to, rituximab (e.g., RITUXAN®; Biogen IDEC, Cambridge, Mass.). In yet another embodiment, phase I (depletion) may be performed by administering a medication which destroys or disables one or more classes of immunoglobulins. An example of such a medication is, but is not limited to, endoglycosidase including EndoS.

In other embodiments, once removed from the patient undergoing treatment, the plasma can be discarded. In these embodiments, the patient undergoing treatment can receive replacement donor plasma. Alternatively, removed blood volume can be replaced with a physiologically acceptable isotonic solution. Examples of solutions suitable for the present invention include, but are not limited to, normal saline solution, isotonic glucose solution, isotonic mannitol solution, isotonic sorbitol solution, isotonic lactose or lactic acid solution (e.g., lactated Ringer's solution) and isotonic glycerol solution. In one embodiment, the blood volume is replaced with a normal saline solution.

In certain embodiments, the patient can be administered various medications immediately before, during, or immediately after apheresis. The term “immediately,” as used herein, will refer to a period of time within no more than 1 hour of the procedure. Examples of medications suitable for administration include, but are not limited to, anticoagulants and neutralizing agents. In some embodiments, a patient can be administered an anticoagulant medication immediately prior to apheresis. In certain embodiments, the anticoagulant medication is selected from sodium citrate, heparin, ximelagatran, argatroban, lepirudin, bivalirudin, warfarin, phenindione, acenocoumarol, phenprocoumon, and combinations thereof. In one embodiment, the anticoagulant medication is sodium citrate. The anticoagulant medication is administered in a pharmaceutically effective amount. As used herein, the term “pharmaceutically effective amount” means the amount of active ingredient that will elicit the biological or medical response of a tissue, system, or animal that is being sought by a clinician. In some embodiments, the pharmaceutically effective amount of sodium citrate is from about 0.1 g/min to about 1 g/min over a period of about 0.5 to about 2 hours. In one embodiment, glucose citrate is administered at a rate of 0.5 g/min over a period of about 1 to about 1.5 hours.

Phase 1 (depletion) of the treatment described herein is followed by Phase 2 (enrichment), which comprises administering to the patient a preparation of immunoglobulins (preferably immunoglobulin G, also known as, and referred to herein interchangeably, as IgG or mixed gammaglobulins) typically administered intravenously (in an approach termed herein as the administration of “intravenous immunoglobulins” or “IVIG”), in an amount sufficient to populate the patients immune system with several hundred million antibodies and achieve a complete restoration of missing or depleted antibodies.

### IVIG Preparations

IVIG preparation suitable for the present invention can be prepared using the following methods. In a preferred embodiment, the resulting preparation will contain at least 20% to at least 45% active immunoglobulins, as determined by assays disclosed herein. In other embodiments, the resulting preparation will contain greater than about 50% active immunoglobulins.

In some embodiments, the highly effective IVIG is purified from other bodily fluids including, but not limited to plasma, blood, serum, synovial fluid, cerebrospinal fluid, colostrum, and nipple aspirates. In one embodiment, the highly effective IVIG is purified from plasma. In a preferred embodiment, the highly effective IVIG is purified from a crude immunoglobulin-containing plasma protein fraction.

In one embodiment, the highly effective IVIG of the present invention is prepared from blood of healthy volunteers, where the number of blood donors is at least about 5 or 10; preferably at least about 100; more preferably at least about 1,000; still more preferably at least about 10,000. In one embodiment, in order to reduce the chances of inadvertent activation of immune reactions in patients receiving the highly effective IVIG, the healthy volunteers are matched by specific characteristics. In one embodiment, the volunteers are age-matched. In another embodiment, the volunteers are matched by their ethnicities. Thus, in one aspect, all volunteers are Caucasians. In another aspect, all volunteers are Asians. In yet another aspect all volunteers are Africans. In still another aspect, all volunteers are Pacific Islanders. In yet another embodiment, the volunteers are matched in a continent-specific manner. Therefore, in one embodiment, all volunteers are North Americans. In another embodiment, all volunteers are South Americans. In another embodiment, all volunteers are Europeans. In another embodiment, all volunteers are Asian. In yet another embodiment, all volunteers are African. In still another embodiment, all volunteers are Australians. In other embodiments, the volunteers are matched by their nationalities.

In one embodiment, the method of purifying highly effective IVIG comprises removal of one or more components of the coagulation pathway from the bodily fluid. Hemostasis is the mechanism by means of which living beings respond to a hemorrhage and involves the participation of two processes that become functional immediately after a lesion and remain active for a long period of time. The first of them is known as primary hemostasis and is characterized by the occurrence of vasoconstriction at the vascular lesion site and platelet aggregate formation. The second one is known as secondary hemostasis, being the phase in which the fibrin clot is formed due to the action of the different coagulation cascade cofactors and proteolytic enzymes, all referred to as coagulation factors. Blood clot formation ending with fibrin formation from fibrinogen hydrolysis due to the action of thrombin. Thrombin is previously formed by proteolytic hydrolysis of an apoenzyme, prothrombin. This proteolysis is carried out by the serine protease FXa, which binds to the surface of the activated platelets and only in the presence of its cofactor, activated coagulation Factor V (FVa), and calcium ions, this serine protease is able to hydrolyze prothrombin. FXa occurs by two separate pathways, the intrinsic pathway and the extrinsic pathway. The intrinsic pathway consists of a series of reactions involving mainly coagulation Factor VIII (FVIII), coagulation Factor IX (FIX) and coagulation Factor XI (FXI), in which each proenzyme is hydrolyzed, yielding its active protease form (FVIIIa, FIXa and FXIa). In the blood coagulation extrinsic pathway, the Tissue Factor (TF) exposed on adventitia cells at the lesion site, binds to circulating coagulation factor VII/activated coagulation Factor VII (FVII/FVIIa) to form the TF::FVIIa complex and, in the presence of calcium, to act as a substrate for FX activation. The extrinsic pathway is currently considered the most relevant pathway in blood coagulation, and it is accepted that in the event of a hemorrhage produced by a vascular lesion, coagulation is triggered due to extrinsic pathway activation involving the interaction of TF with its ligand, FVII/FVIIa.

Therefore, in specific embodiments, the components of the coagulation pathway comprise coagulation Factor V, coagulation Factor VII, coagulation Factor VIII, coagulation Factor IX, coagulation Factor X, coagulation Factor XI, coagulation Factor XII, coagulation Factor XIII and combinations thereof.

Several methods for removal of proteins, including coagulation factors, are known in the art. These include, but are not limited to cryoprecipitation, alcohol precipitation, ultracentrifugation, dialysis, centrifugal filtration, and chromatographic separation, or a combination thereof. Chromatographic separation may include ion exchange chromatography, affinity chromatography, size exclusion chromatography, HPLC, FPLC.

In one embodiment, undesired proteins in the bodily fluid are removed by precipitation. In one aspect, proteins are removed by addition of ammonium sulfate. In another embodiment, undesired proteins are removed by addition of low concentration of polyvalent metal ions such as Ca²⁺, Mg²⁺, Mn²⁺ or Fe²⁺. In another aspect, undesired proteins are removed by the process of floculation involving the addition of polyelctrolytes such as Alginate, carboxymethycellulose, polyacrylic acid, tannic acid, or polyphosphates. In yet another embodiment, undesired proteins are removed by addition of alcohol. In one aspect cols alcohol is added to precipitate undesired proteins.

In one embodiment, the method of purifying highly effective IVIG comprises adding one or more alcohols to the bodily fluid to remove undesired proteins. In one aspect, the addition of one or more alcohols comprises one or more cold alcohol precipitation steps of proteins present in the bodily fluid. Several methods of cold alcohol precipitation are known in the art. A frequently employed method of cold alcohol precipitation is the Cohn-Oncley fractionation, also referred to as 6/9 method (Cohn et al., J. Am. Chem. Soc. 68: 459-475, 1946); Oncley et al., J. Am. Chem. Soc. 71:541-550, 1949)). Another well-employed method of cold alcohol precipitation is the Kistler and Nitschmann ethanol fractionationation (Kistler et al., Vox Sang, 7: 414-424, 1962). Generally, the Kistler and Nitschmann process uses fewer protein precipitation steps and hence less ethanol, and is more cost effective.

In one embodiment, the addition of one or more alcohol leads to the precipitation and removal of undesired proteins from the bodily fluid. Therefore, the addition of alcohol results in enrichment of the IgG in the bodily fluid. In one embodiment, the addition of alcohol results in the bodily fluid containing greater than about 30% IgG. In a preferred embodiment, the addition of alcohol results in the bodily fluid containing greater than about 99% IgG. In specific embodiments, the alcohol added includes, but is not limited to, ethanol, methanol, propanol, butanol, and isoamyl alcohol.

Several steps in the purification of IVIG that seem to have a low likelihood of damage may cause significant reduction of relatively intact IgG or depletion of IgG subclasses. An example of this would be a virus filter that may trap and eliminate large quantities of desired fractions. To avoid the potential loss of active IgG, in one aspect of the invention, the bodily fraction is diluted to reduce the IVIG concentration prior to the filtration step. In another embodiment, the bodily fluid is diluted following the addition of on or more alcohols to remove undesired proteins. In some embodiments, the bodily fluid is diluted at least about 1:1, at least about 1:2, at least about 1:3, at least about 1:4, or at least about 1:10. In some embodiments, the bodily fluid is diluted to a concentration of less than about 1 g/L, less than about 2 g/L, less than about 5 g/L, less than about 10 g/l, less than about 20 g/L, or less than about 50 g/L. In a preferred embodiment, the bodily fluid is diluted to a concentration of less than about 12.5 g/L. In one embodiment, the method of the present invention further comprises addition of one or lubricants to the diluted bodily fluid. In one embodiment, the lubricants is lecithin. In another embodiment, the lubricant is a detergent. Examples of detergent lubricants are well known in the art.

In one embodiment, the method of purifying highly effective IVIG comprises concentrating the bodily fluid by removing water from the bodily fluid. In some embodiments, the bodily fluid is concentrated by using methods well known in the art including, but not limited to, ultracentrifugation, centrifugation, filtration, ultrafiltration, dialysis, and heating. In a preferred embodiment, the bodily fluid is concentrated using an ultrafilter. Filter type has a significant impact on the quality of concentrated bodily fluids obtained by filtration. Some filters produce substantial coagulation and complement activation and cell release, while others appear to reduce the levels of activation markers. Therefore, in one embodiment, conditions for concentrating the bodily fluid are maintained that avoid activation of the complement pathway in the bodily fluid. In one aspect, the condition that avoids activation of the complement pathway comprises a choice of the ultrafilter used for concentrating the bodily fluid.

In one embodiment, the method of purifying highly effective IVIG comprises treating the bodily fluid to eliminate one or more contaminants from the bodily fluid. In one aspect, the one or more contaminants comprise one or more active viral contaminants. In one aspect, the one or more active viral contaminants comprise one or more enveloped virus. In another aspect, the one or more active viral contaminants comprise one or more non-enveloped virus. In another embodiment, the one or more contaminants comprise one or more active microbial contaminants. In yet another embodiment, the one or more contaminants comprise one or more active prions or prion-like contaminants. In one embodiment, elimination of the active viral, microbial or prion contaminants from the bodily fluid involves physical removal of the viral, microbial or prion contaminants. In another embodiment, elimination of the active viral, microbial or prion contaminants from the bodily fluid involves inactivation of the viral, microbial or prion contaminants. A number of methods to eliminate active viral, microbial or prion contaminants from bodily fluids are known in the art including, but not limited to, filtration, ultracentrifugation, chromatographic separation, neutralization mediated by antibodies, and heat inactivation.

In one embodiment, the elimination of one or more active viral, microbial, and prion contaminants from the bodily fluid comprises one or more filtration steps. In one aspect, the one or more filtration steps comprises a pre-filter step. In one aspect, the pre-filter is a 100 nm pre-filter. In another embodiment, the one or more filtration steps comprises a virus filter step. In one aspect the virus filter is a 20 nm virus filter. In another embodiment, the one or more filtration steps comprises one or more sterile filtration steps.

In one embodiment, the method of purifying highly effective IVIG comprises adjusting the pH of the bodily fluid. In one aspect, the pH of the bodily fluid is adjusted to between about 1 and about 10. In one embodiment, the pH of the bodily fluid is adjusted to between about 4 and about 6. In a preferred embodiment, the pH of the bodily fluid is adjusted to about 5.

In one embodiment, the method of purifying highly effective IVIG comprises incubating the bodily fluid at a temperature of between about 20° C. and about 50° C. In one embodiment, the bodily fluid is incubated at room temperature. In a preferred embodiment, the bodily fluid is incubated at a temperature of about 30° C. In one aspect, the bodily fluid is incubated at a temperature of about 30° C. for about 1 week to about 6 weeks. In a preferred embodiment, the bodily fluid is incubated at a temperature of about 30° C. for about 2 weeks.

In one embodiment, the activity of the IVIG is monitored by specific assays. In one aspect, the activity of the IVIG is monitored at the end of each step of the purification process. In another aspect, the activity of the IVIG is monitored at the end of at least the last step of the purification process. In one embodiment, the steps of the purification protocol are determined by assaying the activity of the IVIG at the end of the step and comparing to the activity of the IVIG prior to the start of the step.

In one embodiment, the specific assays to measure IVIG activity are able to measure the state of IgG in the input and output from each process to identify the steps that are damaging the IgG antibodies. The steps that do significant damage or lose key fractions of IgG can generally be replaced with low damage equivalents that maintain safety (virus removal and reduction of irritants that produce side effects) while producing a highly efficient IVIG product. In one embodiment, standard measurement tools to make sure that the ratio by weight of IgG subclasses is maintained through the manufacturing process are used in conjunction with the specific activity assays.

In one embodiment, the activity of the IVIG at the end of each individual step of the purification process is about the same as the activity of the IVIG prior to the start of that step. In one embodiment, the activity of the IVIG at the end of each individual step of the purification process is between at least about 95% and at least about 30% of the activity of the IVIG prior to the start of that step.

In one embodiment, the activity of the IVIG at the end of the purification process is about the same as the activity of the IVIG prior to the start of purification process. In one embodiment, the IVIG preparations have at least 30% active immunoglobulins. In yet other embodiments, the IVIG preparations used in this aspect of the invention have at least 45% active immunoglobulins. The IVIG preparations used in the invention can also have more than 50% active immunoglobulins.

In some embodiments, the suitable immunoglobulin solution or fraction can be obtained from any fractionation with ethanol in the cold which yields sufficiently pure fractions of immunoglobulins. Examples of cold alcohol processes include, but are not limited to, Cohn, Cohn-Oncley, or Kistler-Nischmann fractionation processes. (See Cohn E. J. et al, Preparation and properties of serum and plasma proteins. IV. A system for the separation into fractions of protein and lipoprotein components of biological tissues and fluids, J. Am. Chem. Soc. 1946; 68:459-75 and Oncley, J. L. et al, The separation of the antibodies, isoagglutinins, prothrombin, plasminogen, and beta-1-lipoprotein into subfractions of human plasma, J. Am. Chem. Soc. 1949; 71-541-50). The fractionation can be accomplished, as a way of an example, through selective precipitations in the cold at various ethanol concentrations and pH values. An example of suitable Cohn-Oncley alcohol fractionation process is depicted as follows. Process includes fractionation of plasma into a cryoprecipitate and cryoprecipitate-poor plasma fraction. As is standard in the Cohn-Oncley process, further fractionation of cryoprecipitate yields factor VIII, von Willebrand Factor (vWF) as depicted and which is formulated into a purified product. Fractionation of cryoprecipitate also yields fibrinogen and which is formulated into a purified product.

The Cryoprecipitate-poor plasma fraction is further fractionated into a fraction (Fraction I), a fraction (Fractions II+III), a fraction (Fraction IV) and a fraction (Fraction V). Exemplary components of fractions II+III are IgG, IgM, and IgA (immunoglobulin G, M and A, respectively) and formulated into purified IgG product. Similarly, exemplary components of fraction IV include alpha₁ proteinase inhibitor and anti-thrombin III, generally represented by intermediate. A skilled artisan will easily recognize that selective ethanol fractionation can be done at various % w/w of ethanol, temperature, and pH values. Conditions for protein fractionation suitable for preparation of IVIG can be: about 8 to about 25% ethanol, about −10° C. to about −2° C., at pH of about 5.4 to about 7.4. In other embodiments, immunoglobulin fraction can be obtained by ion-exchange or affinity chromatography, or any other method which yields sufficiently pure fractions of immunoglobulins.

In some embodiments, isolated immunoglobulin preparations are assayed for activity. IVIG preparations can be assayed by the methods employed for determination the amount of immunoglobulin κ1 and immunoglobulin κ2 in the plasma, as described herein. Specifically, the presence of immunoglobulin κ in the plasma sample can be conducted using an affinity chromatography. In some embodiments, protein affinity chromatography will be used. In certain embodiments the protein suitable for use in the methods of the present invention is selected from the group consisting of protein L, protein A, protein G, or a combination thereof. In one embodiments, the plasma sample is analyzed using protein A affinity chromatography. Examples of protein A affinity chromatography columns for use in protein A affinity chromatography herein include protein A immobilized onto a controlled pore glass backbone, including the PROSEP-A™ and PROSEP-vA™ columns (Millipore Inc.); protein A immobilized on a polystyrene solid phase, e.g. the POROS 50A™ column (Applied BioSystems Inc.); or protein A immobilized on an agarose solid phase, for instance the rPROTEIN A SEPHAROSE FAST FLOW™ or MABSELECT™ columns (Amersham Biosciences Inc.).

Affinity chromatography for analyzing an IVIG preparation can be conducted as described herein. Specifically, the solid phase for the protein A affinity chromatography can be equilibrated with a suitable buffer before chromatographic separation of the plasma sample. In certain embodiments, the total amount of immunoglobulins in the IVIG preparation will be quantified using methods generally known to a person of skill in the art and described herein. In some embodiments, the IVIG preparation can be loaded directly onto the equilibrated protein A column. The amount of sample loaded on the column will depend on a number of factors, such as an availability of the sample and column's capacity. In some embodiments, at least about 1 ml of the sample is loaded on the column. In other embodiments, at least about 0.2 ml of the sample is loaded.

After the entire IVIG sample is loaded onto the column, the column can be washed with at least 10-15 column volumes with a wash buffer. The preferred pH of the wash buffer is about 7. After washing the column immunoglobulins are eluted by step pH decreasing of eluting buffer. In some embodiments, the column will be eluted with at least about 1-2 column volumes of the eluting buffer. The preferred pH of the wash buffer is about 5. Immunoglobulins κ1 will elute at this pH. Immunoglobulins κ1 will be collected and quantified using methods generally available to a person of skill in the art and described herein. Immunoglobulins κ2 will not elute at pH 5, and will remain bound to the column. These immunoglobulins can be recovered from the protein A column using about 1-2 column volumes of elution buffer having a low pH, e.g. in the range from about 2 to about 4, and preferably in the range from about 2.3 to about 3.5. In some embodiments, the pH of the elution buffer will be about 3.5. In one embodiment, the pH of the elution buffer is about 2.3.

Once immunoglobulin κ2 fraction is collected from the column, the amount of immunoglobulin κ2 can be quantified using methods described herein and generally known to a person of ordinary skill in the art. In some embodiments, the amount of immunoglobulin κ1 is compared to the amount of total immunoglobulin in the IVIG preparation. In certain embodiments, the IVIG preparation will be deemed suitable for the treatment method of the present invention if the amount of immunoglobulin κ1 in the original sample constitutes at least about 20% of the total immunoglobulins in the sample. In a preferred embodiment, the amount of immunoglobulin κ1 in the IVIG preparation will be at least about 35%. In yet another preferred embodiments, the amount of immunoglobulin κ1 is at least about 45%. In other embodiments, the amount of immunoglobulin κ1 is greater than about 50%.

The immunoglobulins may be of any class, subclass and isotype, including but not limited to IgG, IgM, IgA, IgD and IgE, or mixtures thereof, but preferably are enriched in (i.e., predominately contain) IgG immunoglobulins. Also contemplated for use herein are aqueous solutions containing higher concentrations of IVIG, such as those containing approximately 25%-75% w/v cr w/w IVIG. Substantially pure preparations of the “IgG-fraction of IVIG” are also suitable for use herein; such preparations typically contain greater than about 50% w/v or w/w, preferably greater than 75% w/v or w/w, and more preferably greater than about 90% w/v or w/w, of IgG immunoglobulins in the preparation.

The immunoglobulins, suitably IgG immunoglobulins, may be administered to the patient by any suitable means including intravenous, intra-arterial, intra-muscular, intra-peritoneal, subcutaneous, intra-nasal, inhalatory, per os, per rectum, intra-articular or other appropriate administration routes. In one embodiment, the immunoglobulin is administered intravenously. In certain embodiments, the IVIG administration can be commenced within at least 5 hours of completion of apheresis. In some embodiments, the IVIG is administered within at least 10 hours of completion of apheresis. In yet other embodiments, the IVIG is administered within 24 hours of apheresis. In some embodiments, all of the IVIG is administered at once. In other embodiments, infusion of IVIG is repeated at least once, at least twice, at least three times, at least four times, at least five times, at least six times, at least seven times, at least eight times or at least nine times, after the commencement of IVIG therapy (for a total number of IVIG cycles of one, two, three, four, five, six, seven, eight, nine or ten). In one embodiment, the infused IVIG contains at least 50% of the IgG κ1.

In certain embodiments, the preparation of IVIG is administered in an amount of 0-50 grams per day for a total amount of 2.5-200 grams within 1-10 days. A physician administering the treatment will determine the appropriate dosage of IVIG based on patient's weight, disease or disorder, gender, age, and general health status. A determination of the appropriate dosage will also depend on the activity and quality of IVIG preparations. The dosage may be adjusted and/or lowered after it has been determined that there is minimal variation of the activity across multiple batched of the IVIG preparations. In one embodiment, the preparation of IVIG is administered in an amount of 0-20 grams per day a total amount of 5-80 grams within 2-4 days. In another embodiment, the preparation of IVIG is administered in an amount of 0-10 grams per day a total amount of 8-40 grams within 3 days. In yet another embodiment, the preparation of IVIG is administered in an amount of 0-10 grams per day a total amount of 6.25-40 grams within 4 days. In one embodiment, the administration of IVIG follows a schedule: Day 2—0-2 grams; Day 3—0-4 grams; Day 4—0-5 grams; Day 5—0-7 grams; and Day 6—0-10 grams. In another embodiment, the IVIG is administered according to the following schedule: Day 2—1.25 grams; Day 3—2.5 grams; Day 4—0 grams; Day 5—5 grams; and Day 6—10 grams. In another embodiment, the IVIG is administered according to the following schedule: Day 2—1.25 grams; Day 3—0 grams; Day 4—8.75 grams. In another embodiment, the IVIG is administered according to the following schedule: Day 2—1.25 grams; Day 3—3.75 grams; Day 4—0 grams; Day 5—5 grams. In another embodiment, the IVIG is administered according to the following schedule: Day 2—0 grams; Day 3—10 grams. Other suitable schedules for administering the total amount of IVIG desired over the number of cycles (days) desired are well within the purview and expertise of one of ordinary skill, and can be adjusted by a skilled physician based on the needs of the patient in terms of safety, efficacy and comfort.

In some embodiments, the success of the procedure can be monitored by medical personnel. Generally, a patient's plasma immediately after apheresis will be relatively clear. After the first administration of the IVIG preparation, the patient's plasma will be slightly cloudy. Upon completion of the IVIG administration, the patient's plasma will be clear again. This will generally indicate to the physician that the IVIG therapy has been accepted by the patient's body.

In certain embodiments, patient's response to the treatment can be monitored using analytical tools of the present invention. In some embodiments, patient's response to the treatment can be determined by utilizing the urine assay described herein. In some embodiments, patient's urine will be collected prior to the start of the treatments, and the amount of immunoglobulins light chain will be determined. As the treatment progresses, patient's urine samples can be regularly collected and assayed for the present of immunoglobulins light chain. It is expected that the amount of immunoglobulins light chain will be significantly reduced as the patient is undergoing the treatment of the present invention.

In other embodiments, the patient's response to the treatment can be determined by utilizing the plasma assay described herein. In some embodiments, the patient's plasma will be collected prior to the start of the treatments, and the ratio of immunoglobulins κ1 to κ2 will be determined. As the treatment progresses, the patient's plasma samples can be regularly collected and assayed for the ratio of κ1 to κ2. It is expected that the ratio of κ1 to κ2 will be significantly increased as the patient is undergoing the treatment of the present invention.

In some embodiment, the patient can be administered various medications immediately before, during, or immediately after IVIG infusion. Examples of medications suitable for administration include, but are not limited to, antihistamines and antiinflammatories. In some embodiments, a patient can be administered an antihistamine medication immediately prior to IVIG infusion. In certain embodiments, the antihistamine medication is selected from diphenhydramine, loratadine, Desloratadine, Fexofenadine, Meclizine, Pheniramine, Cetirizine, Promethazine, Chlorpheniramine, levocetirizine, Cimetidine, Famotidine, Ranitidine, Ciproxifan, and Clobenpropit. In one embodiment, the patient is administered a pharmaceutically effective amount of diphenhydramine immediately prior to IVIG administration. In some embodiments, the pharmaceutically effective amount of diphenylhydramine ranges from about 50 mg to about 200 mg. In other embodiments, the pharmaceutically effective amount of diphenhydramine ranges from about 70 mg to about 150 mg. In one embodiment, the patient is administered 100 mg of diphenhydramine. In certain other embodiments, the antiinflamatory medication is a non-steroidal antiinflamatory selected from aspirin, ibuprofen, naproxen, diclofenac, aceclofenac, and licofelone, which are used at amounts that may be titrated for the individual patient and/or at amounts that will be familiar to the ordinarily skilled pharmacist and/or physician.

In some embodiments it is not necessary to identify a dysfunction in the immune system of a mammal before correction of the pathogenic immune response with the process of the present invention. Furthermore, the processes of the present invention unexpectedly provide a sustainable restoration of the patient's immune system. The term “sustainable” is used to mean a period of time ranging from about 3 years to about 25 years. This sustainability is achieved by a radical and complete restoration of the immune system of the patient by the methods disclosed herein. The processes of the present invention unexpectedly prevent the patient's immune system from attacking or rejecting, over time, the components needed to restore the immune system of the patient.

In certain embodiments, the immune system restoration therapy of the present invention can be repeated as desired.

Method of Treatment of a Condition Associated with Autoimmune Abnormality

In another aspect, the present invention is directed to a method of ameliorating, treating, or preventing an abnormal condition associated with a pathological immune response in a patient, using the methods of the present invention such as the Eiger Immune Restoration Process (EIRP). In sonic embodiments, the abnormal condition will be a result of a pathological autoimmune response of the patient to an organ, tissue, cell, molecule, or cellular process or factor. In some embodiments, the abnormal condition resulted from an aberrant autoimmune response of the patient to an angiogenic factor. In these embodiments, pathogenic IgG antibodies are often directed to the positive or negative regulators of angiogenesis. Examples of angiogenesis factors (both positive and negative regulators) are listed in Table 1 below:

TABLE 1
Positive and negative regulators of angiogenesis

| Positive regulators | Negative regulators |
|---|---|
| Fibroblast growth factors | Thrombospondin-1 |
| Placental growth factor | Angiostatin |
| Vascular endothelial growth factor | Interferon alpha |
| Transforming growth factors | Prolactin 16-kd fragment |
| Angiogenin | Metallo-proteinase inhibitors |
| Interleukin-8 | Platelet factor 4 |
| Hepatocyte growth factor | Genistein |
| Granulocyte colony-stimulating factor | Placental proliferin-related protein |
| Platelet-derived endothelial cell | Transforming growth factor beta? |
| growth factor | |
| Angiopoietin 1 | Endostatin |

In certain embodiments, the patient can be subjected to the methods of the present invention in order to prevent the onset of one or more symptoms of the disease or condition. In this embodiment, the patient can be asymptomatic. In certain embodiments, the patient can have a genetic predisposition to the disease. When administered to an asymptomatic patient, or to a patient with a genetic predisposition to a certain disease or condition, the method of the present invention can have a prophylactic effect. In other embodiments, the method of the present invention has a treatment effect. In these embodiments, the patient has been diagnosed with a disease or condition, or has exhibited symptoms characteristic of a particular disease or condition.

The methods of the present invention can be used to ameliorate, treat, or prevent a variety of diseases that have an autoimmune component, particularly one that leads to an angiogenic imbalance, in their etiology. Examples of diseases treatable or preventable by the methods of the present invention include, but are not limited to, acquired haemophilia, Addison's disease, alopecia areata, Alzheimer's Disease, ankylosing spondilitis, antiphospholipid syndrome, aplastic anaemia, asthma (acute or chronic), atherosclerosis, autoimmune gastritis, autoimmune hearing loss, autoimmune haemolytic anaemias, autoimmune hepatitis, autoimmune hypoparathyroidism, autoimmune hypophysitis, autoimmune inner ear disease, autoimmune lymphoproliferative syndrome, autoimmune myocarditis, autoimmune oophoritis, autoimmune orchitis, autoimmune polyendocrinopathy, Bechet's disease, bullous pemphigoid, cardiac infarction, cellulitis, cardiomyopathy, Chagas' disease, chronic inflammatory demyelinating polyneuropathy, Chronic obstructive pulmonary disease (COPD), Churg-Strauss syndrome, coeliac disease, Crohn's disease, CREST syndrome, Degos disease, Dermatomyositis, Diabetes mellitus type 1 (which may be latent autoimmune diabetes in adults or LADA), Dilated cardiomyopathy, Endometriosis, Epilepsy, epidermolysis bullosa acquisita, essential mixed cryoglobulinemia, giant cell arteritis, glomerulonephritis, Goodpasture's syndrome, Graves' disease, graft-versus-host disease (GVHD), host-versus graft disease (HVGD), Guillain-Barré syndrome, Hashimoto's thyroiditis, Hidradenitis suppurativa, idiopathic thrombocytopenic purpura, IgA nephropathy, inflammatory bowel disease, Interstitial cystitis, Kawasaki's disease, Lupus erythematosus, Meniere's syndrome, mixed connective tissue disease, Mooren's ulcer, Morphea, multiple sclerosis, myasthenia gravis, pathologic obesity, pemphigus foliaceous, pemphigus vulgaris, pernicious anaemia, polyarteritis nodosa, polyglandular autoimmune syndrome type 1 (PAS-I), polyglandular autoimmune syndrome type 2 (PAS-2), polyglandular autoimmune syndrome type 3 (PAS-3), polymyositis/dermatomyositis, primary biliary cirrhosis, psoriasis, psoriatic arthritis, Raynaud's syndrome, Reiter's syndrome, rheumatoid arthritis, sarcoidosis, Schizophrenia, scleroderna, Sjogren's syndrome, subacute thyroiditis, sympathetic opthalmia, systemic lupus erythematosus, Takayasu's arteritis, Vasculitis, vitiligo, Vogt-Koyanagi-Harada disease and Wegener's granulomatosis.

The methods of the present invention also can be used to ameliorate, treat, or prevent a variety of neoplastic diseases that have an autoimmune component, particularly one that leads to an angiogenic imbalance, in their etiology. Examples of such neoplastic diseases treatable or preventable by the methods of the present invention include, but are not limited to, carcinomas, sarcomas, leukemias, lymphomas, germ cell tumors and blastomas, particularly non-brain carcinomas and sarcomas. Exemplary tumor/cancer types treatable and/or preventable by the methods of the present invention include, but are not limited to, Acute Lymphoblastic Leukemia, Acute Myeloid Leukemia, Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, Bone Cancer, Osteosarcoma, Histiocytoma, Brain Stem, Glioma, Brain Tumor, Central Nervous System Embryonal Tumors, Cerebellar Astrocytoma, Cerebral Astrocytoma/Malignant Glioma, Craniopharyngioma, Ependymoblastoma, Ependymoma, Medulloblastoma, Medulloepithelioma, Pineal Parenchymal, Supratentorial Primitive Neuroectodermal Tumors, Pineoblastoma, Visual Pathway and Hypothalamic Glioma, Brain and Spinal Cord Tumors, Breast Cancer, Bronchial Tumors, Burkitt Lymphoma, Carcinoid Tumor, Gastrointestinal Carcinoma of Unknown Primary, Embryonal Tumors, Central Nervous System Lymphoma, Cerebellar Astrocytoma, Cerebral Astrocytoma/Malignant Glioma, Cervical Cancer, Chordoma, Chronic Lymphocytic Leukemia, Chronic Myelogenous Leukemia, Chronic Myeloproliferative Disorders, Colon Cancer, Colorectal Cancer, Craniopharyngioma, Cutaneous T-Cell Lymphoma, Mycosis Fungoides, Sézary Syndrome, Embryonal Tumors, Endometrial Cancer, Ependymoblastoma, Ependymoma, Esophageal Cancer, Ewing Family of Tumors, Extracranial Germ Cell Tumor, Extragonadal Germ Cell Tumor, Extrahepatic Bile Duct Cancer, Intraocular Melanoma, Retinoblastoma, Gallbladder Cancer, Gastric Cancer, Gastrointestinal Carcinoid Tumor, Gastrointestinal Stromal Tumor (GIST), Extracranial Germ Cell Tumor, Extragonadal Germ Cell Tumor, Ovarian Germ Cell Tumor, Gestational Trophoblastic Tumor, Glioma, Cerebral Astrocytoma, Hairy Cell Leukemia, Head and Neck Cancer, Liver Cancer, Hodgkin Lymphoma, Hypopharyngeal Cancer, Hypothalamic and Visual Pathway Glioma, Intraocular Melanoma, Endocrine Pancreas Islet Cell Tumors, Kaposi Sarcoma, Kidney Cancer, Langerhans Cell Histiocytosis, Laryngeal Cancer, Leukemia, Acute Lymphoblastic Leukemia, Acute Myeloid Leukemia, Chronic Lymphocytic Leukemia, Chronic Myelogenous Leukemia, Hairy Cell Leukemia, Lip and Oral Cavity Cancer, Liver Cancer, Non-Small Cell Lung Cancer, Small Cell. Lung Cancer, Lymphoma, AIDS-Related Lymphoma, Burkitt Lymphoma, Cutaneous T-Cell Lymphoma, Sezary Syndrome, Hodgkin Lymphoma, Non-Hodgkin Lymphoma, Central Nervous System Lymphoma, Waldenström Macroglobulinemia, Malignant Fibrous Histiocytoma of Bone, Osteosarcoma, Medulloblastoma, Medulloepithelioma, Melanoma, Intraocular Melanoma, Merkel Cell Carcinoma, Mesothelioina, Metastatic Squamous Neck Cancer, Mouth Cancer, Multiple Endocrine Neoplasia Syndrome, Multiple Myelorna/Plasma Cell Neoplasm, Mycosis Fungoides, Myelodysplastic Syndromes, Myelodysplastic/Myeloproliferative Diseases, Myelogenous Leukemia, Myeloid Leukemia, Multiple Myeloma, Myeloproliferative Disorders, Nasal Cavity and Paranasal Sinus Cancer, Nasopharyngeal Cancer, Nasopharyngeal Cancer, Neuroblastoma, Oral Cancer, Oropharyngeal Cancer, Osteosarcoma and Malignant Fibrous Histiocytoma of Bone, Ovarian Cancer, Ovarian Epithelial Cancer, Ovarian Germ Cell Tumor, Ovarian Low Malignant Potential Tumor, Pancreatic Cancer, Pancreatic Cancer, Papillomatosis, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Primary Central Nervous System Lymphoma, Prostate Cancer, Rectal Cancer, Renal Cell Kidney Cancer, Renal Pelvis and Ureter Transitional Cell Cancer, Respiratory Tract Cancer, Retinoblastoma, Rhabdomyosarcoma, Salivary Gland Cancer, Ewing Family Sarcoma, Kaposi Sarcoma, Soft Tissue Sarcoma, Uterine Sarcoma, Sézary Syndrome, Non-melanoma Skin Cancer, Merkel Cell Carcinoma, Small Intestine Cancer, Squamous Cell Carcinoma, Stomach Cancer, Cutaneous T-Cell Lymphoma, Testicular Cancer, Throat Cancer, Thymoma and Thymic Carcinoma, Thyroid Cancer, Transitional Cell Cancer of the Renal Pelvis and Ureter, Gestational Trophoblastic Tumor, Urethral Cancer, Endometrial Uterine Cancer, Uterine Sarcoma, Vaginal Cancer, Vulvar Cancer, Waldenström Macroglobulinemia and Wilms Tumor.

Immunoglobulin (Ig) has five fractions (IgM, IgG, IgA, IgD, and IgE). For most of the diseases listed herein that are advantageously treated using the methods of the present invention, IgG administration (in the form of IVIG) is generally sufficient for the second phase of the treatment methods of the present invention. Without wishing to be bound by theory, this is thought to be because healthy IgG is a therapeutic mediator for the other fractions of Ig and can also trigger the complement system. Healthy IgG can indirectly stimulate the production of critical immune system proteins like interleukins, which in themselves can have therapeutic effects in treating certain of the diseases and disorders discussed herein. In other embodiments, however, some of the diseases listed herein may require that IgG be supplemented with IgM, IgA, IgD, and/or IgE during the phase 2 infusion portion of the methods of the present invention. In addition, patients that have insufficient healthy white blood cells, particularly B-cells, may need blood transfusions, bone marrow transplants or other therapies prior to treatment with the methods of the present invention, e.g., EIRP.

Some pathogenic IgG mediated conditions are caused by the aberrant immune response and destruction or disabling of antiangiogenic factors. Many of these diseases/conditions listed above are generally agreed to be auto-immune in nature by people skilled in the art. Other diseases/conditions in this category that are treatable with EIRP include Atherosclerosis (Cardio-vascular Disease), Age-related Macular Degeneration, Diabetic Retinopathy, Neovascular Glaucoma, Hemangiomas, Diabetic Ulcers, Alzheimer's Disease Diabetes and a variety of benign skin growths. Other pathogenic IgG-mediated conditions are caused by the blocking of normal angiogenesis by the destruction or disabling of antiangiogenenic factors, thus promoting premature degeneration of body functions or delaying healing following damage or disease. The EIRP treatment can, in some patients with pathogenic IgG antibodies directed at anti-angiogenesis factors, provide relief from degeneration and promote healing after damage from many conditions/diseases including ageing and stroke. In some embodiments, the aberrant immune response is to an antiangiogenic factor. In one embodiment, the angiogenic disorder is a result of the aberrant autoimmune response of the patient to angiostatin.

In addition, the methods of the present invention can control uncontrolled growth associated with non-malignant or pre-malignant conditions, and other disorders involving inappropriate cell or tissue growth resulting from pathogenic autoantibodies (particularly IgG autoantibodies). This includes diseases/conditions with vascularized tumors or neoplasms or angiogenic diseases. In other embodiments, the method of the present invention can be used to mitigate the immune response to organ transplantation, before and after the transplant surgery, to increase the likelihood that the transplant will not be rejected. In other embodiments, the method of the present invention is useful for treatment or prevention of any disease listed or any other disease/condition found to be mediated by pathogenic IgG antibodies.

In some embodiments, surgery may be required prior to treatment with the method of the present invention. Generally, the surgery will be required to remove very large tumors (over 0.5 kg), or to repair major damage to critical body system. A physician will need to assess a general health of the patient to determine an appropriate course of treatment necessary prior to commencement of the immune system restoration therapy of the present invention. Generally, chemotherapy and radiation therapy should not be required, although can be administered to the patient based on the physician's evaluation of patient's health and condition. Preferably, critical body systems (e.g. liver, kidney, bladder, and bowel) of patients chosen for treatment with the method of the present invention will be able to sustain life including circulation, breathing, nutrition intake and waste removal. In some embodiment, a surgery may be required after the completion of the immune system restoration therapy of the present invention to repair damage caused by the disease.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Protein A Affinity Purifications

Purification of IgG from plasma samples (1 ml each) was performed by passing the plasma over protein A immobilized on Sepharose. Individual affinity columns were prepared by washing with PBS, followed by a mock elution with 0.1 M glycine-HCl (pH 3.0), and then were equilibrated with PBS buffer at pH 7.0 (binding buffer). Plasma sample was mixed with an equal volume of binding buffer and passed over the column with flow rate 0.2 ml/min. Unbound material was removed by washing with binding buffer. Bound IgG k1 was eluted in 1-ml fractions by using 0.1 M ammonium bicarbonate buffer (pH 5.0). Bound IgG k2 was eluted in 1-ml fractions by using 0.1 M glycine-HCl buffer (pH 3.0) The fractions were read at OD₂₈₀, and fractions (≧0.1) were pooled. The protein concentration was determined by taking the absorbance value at OD₂₈₀ and using an extinction coefficient of 13.6 for a 1.0% solution. The purity of the IgG preparations was assessed by SDS-polyacrylamide gel electrophoresis.

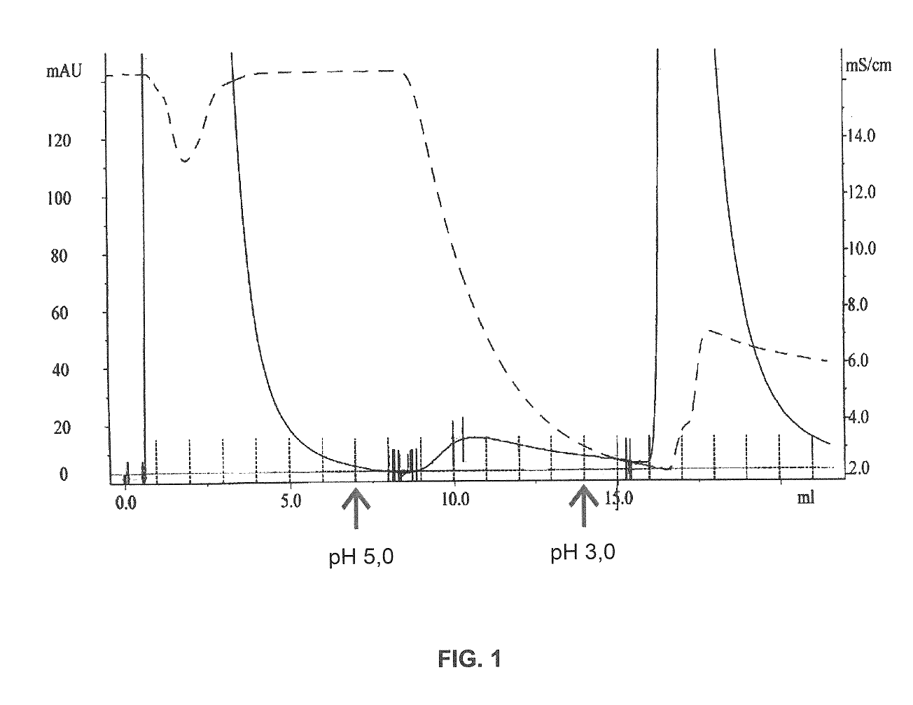

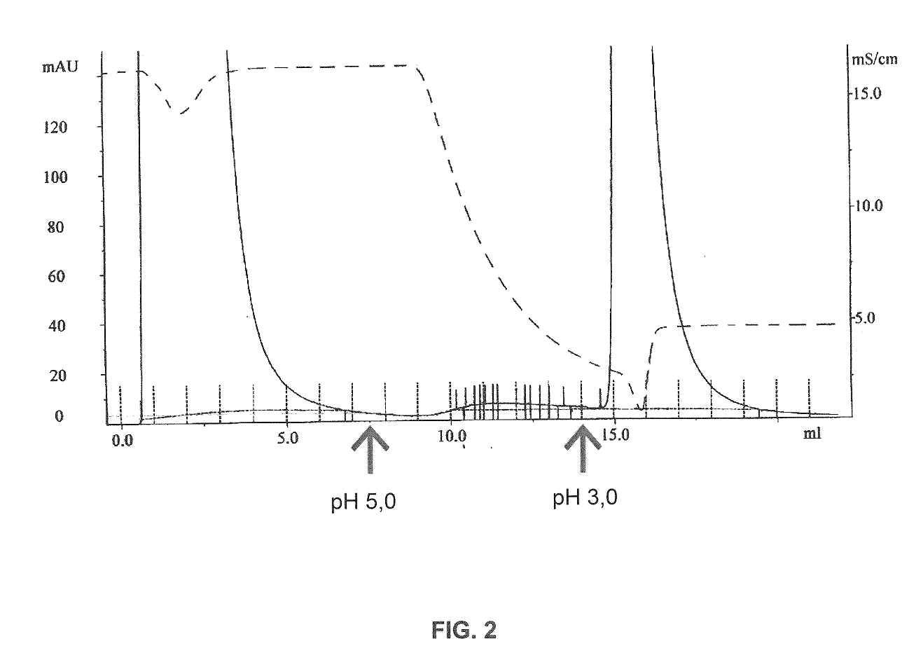

The results of the process described above are presented on FIGS. 1 and 2, wherein FIG. 1 shows an affinity chromatography diagram of an analyzed plasma sample from a healthy person and FIG. 2 shows an affinity chromatography diagram of an analyzed plasma sample from a cancer patient. The figures illustrates that determination of the κ1 to κ2 ratio make it possible to evaluate an immune status of a person.

### Example 2

### Protein L Affinity Purification of Immunoglobuline Light Chains from Urine Samples

Concentration of IgG kappa light chains from urine samples (100 ml each) was performed by passing the urine, equilibrated with PBS pH 7.2 overnight over protein L immobilized on Sepharose. Urine sample was passed over the column with flow rate 2 ml/min. Unbound material was removed by washing with 10 column volumes of binding buffer. Bound IgG kappa light chains were eluted in 0.2-ml fractions by using 0.1 M glycine-HCl buffer (pH 3.0). The fractions were read at OD₂₈₀, and fractions (≧0.1) were pooled. The protein concentration was determined by taking the absorbance value at OD₂₈₀ and using an extinction coefficient of 13.6 for a 1.0% solution.

Urine samples from four patients with various immune disorders were subjected to the analytical procedure described herein prior to and after the treatment of these patients using the treatment methods of the invention described herein. As a control, urine samples from a healthy human were collected and analyzed using the procedure described herein. Results of analysis of urine samples from various individuals are summarized in Table 11.

TABLE 11

24 hours secretion of kappa
light chains in urine(mg)

| Patient diagnosis | before treatment | after treatment |
|---|---|---|
| normal control | 3.6 | Not treated |
| rheumatoid arthrities | 46.2 | 12.4 |
| multiple sclerosis | 109.3 | 17.5 |
| lupus erythemathosus | 77.6 | 10.3 |
| hepatocarcinoma | 140.8 | 15.2 |

### Example 3

### A Design for an Effective IVIG Manufacturing Process

Manufacturers will have many process steps in common although there will be some differences between manufacturers. The standard IVIG manufacturing process described below contains the steps commonly used:
a. Removal of Factor VIII and Factor IX using cryoprecipitation and ion exchange.
b. A series of cold alcohol processes (Cohn and Oncley cold ethanol process or variants including the Kistler & Nitschmann cold ethanol fractionation process) and absorption that results in a solution containing greater than 99% IgG.
c. A series of steps using low pH (<5.0), high temperature incubation (>30° C.) and harsh chemicals including solvents and detergents.
d. Some manufacturers use a small amount of detergent (lubricant) and a filter that will remove any remaining viruses.
e. Concentration by ultrafiltration to remove water.
f. A last sterile filtration to remove microbial contaminants.
g. Adjust to proper pH (typically 4-6) and add stabilizers and fill.
h. Incubation at 30° C. for 2 weeks.

By examining the damage to IgG after each step using the analytical method for plasma described above, it is possible to identify the steps causing the largest amount of damage to the IgG during processing. For example, if the donor plasma assay shows that x % of the IgG is highly glycosylated, the goal is that the final IVIG product should assay to no less than 0.85x %. Using healthy donors, it should be possible to produce IVIG that has over 30% highly glycosylated IgG using the assay technique described in the analytical method described above. The steps that are unlikely to produce significant damage are steps “A”, “B”, “F”, “G” and “H”, Step “C” will generally produce significant damage. The virus filtration step (step “D”) makes the step “C” processes unnecessary. Step “D” may produce several problems. Undamaged IgG at normal manufacturing concentrations will tend to “clump” such that it cannot pass through a virus filter. This would eliminate most of the critical IgG needed for full efficacy. Also, some IgG bands may be lost in the filter at high concentrations. The virus filter performs better when the IgG is diluted to less than 5 g/L. At this concentration, very low losses of IgG will be observed. Step “E” may be accomplished using several different approaches. At least one of the available techniques, a filter membrane with recirculating IVIG mixture washing across it, may initiate complement activation and increasing the risk of side effects with the resulting IVIG. This situation is less frequent with damaged IgG but common with the undamaged IgG that should result from an improved manufacturing process. Should this be observed, another membrane material or an alternate method to remove excess water should be chosen.

A reworked IVIG manufacturing process at an individual manufacturing plant will have characteristics that may be unique to that plant. The manufacturing schematic design is one example of a process that can produce IVIG that is both safe and effective.
a. Removal of Factor VIII and Factor IX using cryoprecipitation and ion exchange.
b. A series of cold alcohol processes (Cohn and Oncley cold ethanol process or variants including the Kistler & Nitschmann cold ethanol fractionation process) and absorption that results in a solution containing greater than 99% IgG.
c. Dilute the mixture to less than 12.5 g/L and add detergent as lubricant prior to filtration.
d. A filter step using a 100 nm pre-filter and a 20 nm virus filter that will remove both enveloped and non-enveloped viruses.
e. Concentration by ultrafilter to remove water taking care in the choice of filter material to avoid complement activation.
f. A last sterile filtration to remove microbial contaminants.
g. Adjust to proper pH (4-6) and add stabilizers and fill.
h. Incubation at 30° C. for 2 weeks.

The treatment of most cancers and other auto-immune diseases is possible using small dosages of IVIG that is highly glycosylated, comparable to that found in the plasma of healthy donors. The preferred treatment regime uses a two phase process over multiple days. The first phase each day is depletion of the patient's plasma using an aphaeresis device. For an adult patient, 500-800 ml of plasma is removed and discarded each day. Depletion of plasma while maintaining blood volume with normal saline solution causes a “squeezing” of the organs and interstitial spaces. Defective immune complexes, waste products and destructive proteins are drawn into the blood stream. The second phase each day is enrichment of the patient's immune system with IVIG. The dosage on the first day needs to be only 1-2 g of active IVIG for an adult. The dosage on each of the subsequent days is 5-7 g for an adult. To avoid possible allergic reaction, it is desirable to pre-medicate the patient with 20 mg of IV Benadryl each day. Two days of treatment should be sufficient for most patients. An additional day or two may marginally improve the odds of successful treatment. A two day treatment protocol with 2-4 weeks of rest and then an additional two days of treatment should maximize the chance for successful outcome. The obvious alternative to using IVIG is donor plasma. Positive outcomes with the two-phase plasma treatment protocol are possible but much less likely than with IVIG. The results following the two-phase IVIG treatment protocol will vary by disease, individual and general health prior to treatment.

Immediately after treatment and for a period of 2-8 weeks, most inflammatory body processes are halted. Patients generally feel more energetic. Pain is lessened. Some patients experience signs of general rejuvenation but this effect is probably not long lasting. The treatment appears to hold for more than 3 years without additional treatments. No data is currently available beyond 3 years from treatment.

Data from over 100 solid tumor cancer patients indicate that tumors tend to decline in size by 10-20% per month following treatment. By 6-8 months after treatment, tumors should not be visible on scans. Small cancer clusters (<2 mm) that do not require their own blood vessels remain after 8 months but do not grow. Patients with many common cancer types follow this pattern. The auto-immune conditions treated successfully include rheumatoid arthritis, lupus, psoriasis, multiple sclerosis, diabetes and Alzheimer's.

With highly effective IVIG, it is possible to treat these same patients subcutaneously or intramuscularly. The site of injection should be near the tissue that is problematic for the specific condition. It should also be near major lymph system circulation points. It appears likely that many or even most auto-immune conditions will be treatable with the modified IVIG protocol disclosed herein.

### Example 4

### Treatment of Cancer Patients, 4-Day Cycle

Patients were identified as being afflicted with certain non-brain solid tumors, and traveled to a treatment facility associated with Eiger Health to be evaluated for, and receive, treatment using the Eiger Immune Restoration Process (“EIRP”). The treatment proceeded according to the following exemplary 4-day schedule (although it must be noted that adjustments to this schedule can be made if necessary based on patient necessity; such adjustments to this exemplary schedule, if any, are noted in the patient results tables shown below):

Day 0—Before Travel and Treatment

0A Obtain a complete medical history from the patient or the patient's physician.

0B Speak with the patient (and the patient's physician whenever possible) to be sure that the patient is a good candidate for treatment, understands the risks and has reasonable expectations following treatment.

The patient should bring a relative or friend to be with them during travel and treatment.

Once treatment has begun, the patient should not drive until at least 24 hours after the last treatment day.

Answer the patient's questions, obtain informed consent, and establish a desired schedule for treatment. Document the conversation and any questions that arose.

0C Confirm schedule and availability of personnel and facilities for treatment.

### Equipment and Medications for EIRP

The type of devices, supplies and medications used are approved and in common use worldwide. The actual devices used for treatment in Lithuania and Russia are sourced from Russia, Europe and the US:

1. A single-needle membrane plasmapheresis device manufactured by BIOTECH-M in Moscow Russia with model designation GEMOS. The device uses a membrane to separate cellular material from the patient's blood which is immediately returned to the patient while eliminating plasma with molecules including circulating immune complexes. The unit replaces the plasma taken with normal saline solution to maintain blood volume in circulation.

2. Normal saline solution (0.9% sodium chloride in water) packaged for intravenous injection.

3. “Glugicir” packaged for intravenous injection. Glugicir is a sterile, apyrogenic, glucose and sodium citrate solution in water for injections (till 1 liter) that contains sodium hydrocitrate disubstituted for injections—20 g, glucose (in recount on anhydrous)—30 g. This is used with plasmapheresis as an anticoagulant.

4. Calcium Gluconate solution (1.0 g in 10 ml) packaged for intravenous injection. This is used at the end of the plasmapheresis procedure to neutralize the acidity of the Glugicir.

5. Benadryl (Diphenhydramine) solution (100 mg in 2.0 ml) packaged for intravenous injection. This medication is intended to prevent or reduce some of the patient discomfort that can be associated with the infusion of immunoglobulin.

6. Immunoglobulin (gamma globulin) solution (1.25 in 25 ml) packaged for intravenous injection.

7. Assorted sterile bandages and other supplies associated with plasmapheresis and IV administration.

Patient Treatment with Eiger Immune Restoration Process (EIRP)

Day 1—Arrival and Brief Examination After Travel

1A Inventory and check status of all devices, medications, supplies and facilities to be used during treatment.

1B Reconfirm schedule and availability of personnel and facilities for treatment.

There is a physician and one other trained person available at all times during treatment. The second person could be a physician or a nurse that is fully qualified to establish an IV line, administer IV medications, run the specific plasmapheresis device and monitor patient progress.

1C Meet patient and conduct a brief examination including:

A. Assessment of general patient health

B. Major body systems

C. Cancer site(s)

D. Determine if there is any issue that would make treatment of the patient unsafe or unwise at this point.

E. Identify any special issues and finalize the plan for the patient's treatment

F. Document the results of the exam.

G. Review the treatment plan including risks with the patient and have informed consent document signed for treatment to proceed.

H. Allow the patient to rest after travel.

Days 2, 3, 5 and 6—Treatment Days

2A Recheck inventory and check status of all devices, medications, supplies and facilities to be used during treatment. Proceed when all required elements are ready for treatment. Begin documentation of day's activities.

2B Ask the patient if there have been any changes in health since arrival and adjust treatment plan as required.

2C Establish double-needle IV line for plasmapheresis.

2D Establish the plasmapheresis connections for normal saline solution and sodium citrate.

2E Run the plasmapheresis device lines until satisfied that the device, filter and all lines have been properly prepared:

A. Flow rate for sodium chloride solution matched to plasma elimination rate to keep blood volume as constant as possible

B. Flow rate for sodium citrate (Glugicir) set to 0.5 g/minute

C. Pumping correctly

D. Membrane filter functioning correctly

E. Blood flowing and no bubbles in lines

2F Begin plasmapheresis procedure to remove approximately 0.6 liters of plasma over a period of 1-1.5 hours. The plasma is collected and discarded.

2G Monitor the patient and plasmapheresis device making adjustments as required for patient comfort and plasmapheresis device function.

2H When the target amount of plasma has been removed, infuse 10 ml of Calcium Gluconate solution to neutralize the blood acidity caused by the sodium citrate.

Note—Administration of Calcium Gluconate will cause a warming sensation at the IV site and internally in the patient. The patient should be alerted to this natural and harmless reaction prior to infusion.

2I Disconnect the IV line from the plasmapheresis device to the patient and check the patient's progress for a minimum of 15 minutes after the completion of plasmapheresis,

The patient should not drive themselves until at least 24 hours after the last day of treatment.

Note: Following plasmapheresis, the following signs/symptoms are normal:

A. Mild light headedness or dizziness for up to 2 hours.

B. Mild warm and cold spots around the body

C. A lowering of pain in joints, back and in the area of cancer tumors.

D. An improved sense of well being.

E. Sleepy or tired.

2J While patient is being observed following plasmapheresis, prepare the immunoglobulin, normal saline solution and IV line for infusion of immunoglobulin.

The immunoglobulin dosages/times for each treatment day are as follows:
Day 2—1.25 grams in 250 ml of normal saline solution over 45 minutes
Day 3—2.50 grams in 250 ml of normal saline solution over 45 minutes
Day 4 rest day no treatment
Day 5—5.0 grams in 500 ml of normal saline solution over 1 hour
Day 6 10. grams in 500 ml of normal saline solution over 1.25 hours

2K Prepare a syringe for 1V infusion of Benadryl solution (100 mg in 2 ml)

Connect the IV line with normal saline to the patient IV connector and infuse the Benadryl to reduce possible allergic reaction to IVIG. When complete, remove the Benadryl syringe

Note—many patients may fall into a comfortable sleep for 10-40 minutes and some patients may feel some anxiety after Benadryl administration.

2L Attach the IVIG line for immunoglobulin infusion and begin administration at the rate shown in “2J” above.

Watch carefully for any allergic reaction. In the event of any serious reaction, cease IVIG administration immediately but continue to infuse normal saline solution.

2M When WIG infusion is completed, flush the IV catheter with 5 ml of normal saline solution.

Remove the IV catheter and clean and bandage the IV site.

2N Observe the patient for a minimum of 15 minutes for any remaining signs of adverse reactions.

During the observation, quickly re-examine the patient's health status and document any signs/symptoms including the patients comments on changes observed.

2O Only on Day 6 (last day of treatment): Reexamine patient, review instructions and expectations and provide written follow-up plan.

2P Patients may leave the treatment facility, preferably with family or friend.

Patients should not drive themselves until 24 hours after the treatment is completed on day 6.

Day 4—Patient Rest Day

4A Patient should be contacted twice during the day (morning and afternoon).

The patient contacts have three purposes:

A. Determine whether the patient has had any adverse reactions to treatment.

B. Answer any questions that the patient may have.

C. Identify any new health events that could impact patient safety or treatment outcome.

4B Document patient progress and issues.

4C Adjust the remaining treatment schedule, if needed.

4D Reconfirm schedule and availability of personnel and facilities for treatment.

Results of treatment of five representative human cancer patients are shown in Tables 2-6 below. In each table, “EIRP” treatment refers to treatment with one embodiment of the methods of the present invention (an embodiment that is referred to herein as the “Eiger Immune Restoration Protocol” or “EIRP”).

TABLE 2
(Patient #1; human)

| | |
|---|---|
| Age and sex | 73, male |
| Condition or disease | Cancer of the lung and lymph nodes. The patient has only |
| | one kidney and a history of severe atherosclerosis. |
| Severity | On oxygen 24/7. Left lung closed by large tumor around |
| | bronchus. Patient was constantly tired and unable to work (artist). |
| Prior treatment | Radiation and chemotherapy for seven weeks |
| Results achieved in prior treatment | Ineffective - lung cancer grew and spread to lymph nodes |
| Date treated with EIRP | June 2009 |
| EIRP treatment | 5 days with 4 treatments (The patient rested with no |
| | treatment on day #3). |
| Results Achieved with EIRP | Blockage of left lung bronchus relieved on day two of |
| | treatment. Oxygen requirement dropped to 2 hours a day |
| | immediately (except for plane flight which did require |
| | oxygen). |
| | Tumor size measured by CT scan at treatment plus 30 and |
| | 60 days shows consistent decline at a rate of approximately |
| | 20% per month. |
| | On physical exam by his physicians, the patient has full air |
| | flow in both lungs. |
| Complications and side effects | There were no adverse effects observed related to the |
| | treatment. |
| | Two weeks after the EIRP treatment, the patient was |
| | hospitalized for 10 days. In the opinion of three of his |
| | regular doctors, this related to damage done to his left lung |
| | caused by the previous 7 weeks of radiation and |
| | chemotherapy |
| Current condition | At 90 days after EIRP treatment, the patient is symptom |
| | free and working daily without oxygen. His energy level is |
| | significantly higher. |
| | No accurate measurement of the effect on atherosclerosis |
| | has been possible yet due to new limitations on the use of |
| | angiograms in the US. |

TABLE 3
(Patient #2; human)

| | |
|---|---|
| Age and sex | 70, female |
| Condition or disease | Cancer of the endometrium. The patient has |
| | severe. atherosclerosis and type 2 diabetes. |
| Severity | Patient was constantly tired and unable to do |
| | home work |
| Prior treatment | No prior treatment |
| Results achieved in prior treatment | |
| Date treated with EIRP | November 2008 |
| EIRP treatment | 2 days with 2 treatments and 3 days with 3 |
| | treatments after 2 month |
| Results Achieved with EIRP | Improvement of physical productivity on day two |
| | of treatment. After 4 month from the beginning |
| | of treatment both utherus and endometrium |
| | volume decreased by 15% |
| | On physical exam by his physicians, the patient |
| | has stable state of gynecological disease. |
| Complications and side effects | There were no adverse effects observed related to |
| | the treatment. |
| Current condition | At 10 month after EIRP treatment, the patient is |
| | symptom free and all other diseases are in stable |
| | state. His energy level is significantly higher. |

TABLE 4
(Patient #3; human)

| | |
|---|---|
| Age and sex | 71, male |
| Condition or disease | Cancer of the esophagus and lymph nodes. |
| Severity | Patient was starved because of unabling eating |
| Prior treatment | Radiation and chemotherapy for two weeks |
| Results achieved in prior treatment | Ineffective - esophagus cancer grew and spread to lymph |
| | nodes |
| Date treated with EIRP | November 2008 |
| EIRP treatment | 5 days with 5 treatments |
| Results Achieved with EIRP | Blockage of esophagus relieved on day two of treatment. |
| | Patient started consumption of normal food. CT results |
| | showed 15% decrease in volume of tumor one month after |
| | treatment |
| Complications and side effects | There were no adverse effects observed related to the |
| | treatment. |
| | Five weeks after the EIRP treatment, the patient in stressful |
| | situation after consumption of 200 ml of vodka(40% |
| | alcohol) again lost the possibility of eating. After one week |
| | he was operated to install esophagostoma. |
| Current condition | Because of postoperational complications patient died |

TABLE 5
(Patient #4; human)

| | |
|---|---|
| Age and sex | 49, female |
| Condition or disease | Left salivary gland cancer with lung methastasys and the |
| | history of disease from 1986 The patient was undergo |
| | twice (1986 and 2003) full course of combinatorial |
| | treatment, including chemo- and radiotherapy, without |
| | clinical response |
| Severity | Continuous pain in the mouth. Chronic cough. Patient |
| | was constantly tired and unable to work (housewife). |
| Prior treatment | Radiation and chemotherapy for 20 weeks |
| Results achieved in prior treatment | Ineffective - lung methastases grew and spread |
| Date treated with EIRP | November 2008 |
| EIRP treatment | 5 days with 5 treatments |
| Results Achieved with EIRP | Disease stabilized, pain in the mouth disappeared. Cough |
| | minimized. CT 2 month after treatment showed 20% |
| | decrease of metasthases size and number. |
| Complications and side effects | There were no adverse effects observed related to the |
| | treatment. |
| Current condition | At 12 month after EIRP treatment, the patient is pain free |
| | and her energy level is significantly higher. |

TABLE 6
(Patient #5; human)

| | |
|---|---|
| Age and sex | 39, female |
| Condition or disease | Breast cancer of the right mammary gland. . |
| Severity | The tumor size was 26.8 mm × 20.7 mm × 22.3 mm, and was |
| | constantly growing. Patient was depressed and unable to |
| | work (medical sister). |
| Prior treatment | No prior treatment |
| Results achieved in prior treatment | Patient refused operation and chemotherapy |
| Date treated with EIRP | June 2008 |
| EIRP treatment | 5 days with 5 treatments |
| Results Achieved with EIRP | Growing of tumor stops on day two of treatment. Multiple |
| | USI investigations(practically every month) don't show |
| | any progressing of disease |
| Complications and side effects | There were no adverse effects observed related to the |
| | treatment. |
| Current condition | At 16 month after EIRP treatment, the patient is symptom |
| | free and working daily. Her depression disappeared |

### Example 5

### Treatment of Cancer Patients, 3-Day Cycle

Patients were identified as being afflicted with certain non-brain solid tumors, and traveled to a treatment facility associated with Eiger Health to be evaluated for, and receive, treatment using the Eiger Immune Restoration Protocol (“EIRP”). The treatment proceeded according to the following exemplary 3-day schedule (although it must be noted that adjustments to this schedule can be made if necessary based on patient necessity; such adjustments to this exemplary schedule, if any, are noted in the patient results tables shown below):

### Equipment and Medications for EIRP

The type of devices, supplies and medications used are as described in Example 3.

Patient Treatment with Eiger Immune Restoration Protocol (EIRP)

Patient's evaluation, preparation, and plasmapheresis is conducted as described in Example 3.

The IVIG is administered according to the method of Example 3, but the dosage/times of immunoglobulin are changed as follows:
Day 2—2.0 grams in 250 ml of normal saline solution over 45 minutes
Day 3—2.50 grams in 250 ml of normal saline solution over 45 minutes
Day 4—rest day, no treatment
Day 5—5.0 grams in 500 ml of normal saline solution over 1 hour.

### Example 6

### Treatment of Cancer Patients, 2-Day Cycle

Patients were identified as being afflicted with certain non-brain solid tumors, and traveled to a treatment facility associated with Eiger Health to be evaluated for, and receive, treatment using the Eiger Immune Restoration Protocol (“EIRP”). The treatment proceeded according to the following exemplary 2-day schedule (although it must be noted that adjustments to this schedule can be made if necessary based on patient necessity; such adjustments to this exemplary schedule, if any, are noted in the patient results tables shown below):

### Equipment and Medications for EIRP

The type of devices, supplies and medications used are as described in Example 3.

Patient Treatment with Eiger Immune Restoration Protocol (EIRP)

Patient's evaluation, preparation, and plasmapheresis is conducted as described in Example 3.

The IVIG is administered according to the method of Example 3, but the dosage/times of immunoglobulin are changed as follows:
a Day 2—4.0 grams in 250 ml of normal saline solution over 1 hour.
a Day 3—rest day, no treatment.
Day 5—6.0 grams in 500 ml of normal saline solution over 1.5 hour.

### Example 7

### Treatment of Cancer Patients, 1-Day Cycle

Patients were identified as being afflicted with certain non-brain solid tumors, and traveled to a treatment facility associated with Eiger Health to be evaluated for, and receive, treatment using the Eiger Immune Restoration Protocol (“EIRP”). The treatment proceeded according to the following exemplary 1-day schedule (although it must be noted that adjustments to this schedule can be made if necessary based on patient necessity; such adjustments to this exemplary schedule, if any, are noted in the patient results tables shown below):

### Equipment and Medications for EIRP

The type of devices, supplies and medications used are as described in Example 3.

Patient Treatment with Eiger Immune Restoration Protocol (EIRP)

Patient's evaluation, preparation, and plasmapheresis is conducted as described in Example 3.

The IVIG is administered according to the method of Example 3, but the dosage/times of immunoglobulin are changed as follows:
Day 2—10 grams in 500 ml of normal saline solution over 2 hours.

As those of ordinary skill will appreciate, similar or analogous schedules can be devised to treat patients over a five-day cycle, a six-day cycle, a seven-day cycle, an eight-day cycle, a nine-day cycle, a ten-day cycle, etc., based on the ordinary skill of the practicing physician in view of the patient's clinical presentation and needs (e.g., comfort, therapeutic efficacy, safety, etc.).

### Example 8

### Treatment of Autoimmune Disorder Patients

Patients were identified as being afflicted with certain autoimmune disorders, and traveled to a treatment facility associated with Eiger Health to be evaluated for, and receive, treatment using the EIRP as outlined in Example 3 (although as one of ordinary skill will recognize, the treatment schedules outlined in Examples 2-4 may similarly or alternatively be used). Results of these treatments are shown in Tables 7-10 below.

TABLE 7
(Patient #6; human)

| | |
|---|---|
| Age and sex | 53, male |
| Condition or disease | MS with history at least 12 years |
| Severity | Practically no movement of legs. Big depression |
| Prior treatment | All known methods of treatment of MS including |
| | interferon- |
| Results achieved in prior treatment | Ineffective - disability of nervous system continuously |
| | increased |
| Date treated with EIRP | June 2008 |
| EIRP treatment | 5 days with 5 treatments |
| Results Achieved with EIRP | Improvement in walking on day two of treatment. |
| | Depression disappeared |
| | On physical exam by his physicians, the patient has |
| | dramatic improvement in his ability to walk. |
| Complications and side effects | There were no adverse effects observed related to the |
| | treatment. |
| | Two weeks after the EIRP treatment, the patient was |
| | hospitalized for 10 days. In the opinion of three of his |
| | regular doctors, this related to damage done to his left lung |
| | caused by the previous 7 weeks of radiation and |
| | chemotherapy |
| Current condition | At 12 month after EIRP treatment, the patient is |
| | continuously improving his physical state. His energy level |
| | is significantly higher. |

TABLE 8
(Patient #7; human)

| | |
|---|---|
| Age and sex | 56, female |
| Condition or disease | Rheumatoid arthritis. The patient has periodic |
| | inflammation in left knee, which became swollen, warm, |
| | painful and stiff |
| Severity | Disease started 11 years ago. Sometimes patient was |
| | unable to walk. |
| Prior treatment | Corticosteroids and NSAID for 11 years |
| Results achieved in prior treatment | Ineffective - inflammation still persisted, even in cases |
| | when side effects of corticosteroid administration |
| | appeared. |
| Date treated with EIRP | January 2009 |
| EIRP treatment | 5 days with 5 treatments |
| Results Achieved with EIRP | Inflammation symptoms relieved on day fore of treatment. |
| | Patient starts to walk without stick. |
| Complications and side effects | There were no adverse effects observed related to the |
| | treatment. |
| Current condition | At 5 month after EIRP treatment, the patient has recurrence |
| | of pain in her knee, but in this case inflammation was |
| | effectively suppressed by administration of NSAID |

TABLE 9
(Patient #8; human)

| | |
|---|---|
| Age and sex | 63, female |
| Condition or disease | Systemic lupus erythematosus. The patient has damaged |
| Severity | kidneys and lungs |
| | Disease is diagnosed 8 years ago, but problems with lungs |
| | started more than 15 years ago. Patient was constantly |
| | tired and unable to work |
| Prior treatment | Corticosteroids and immune-suppressants for 8 years |
| Results achieved in prior treatment | Ineffective - disease constantly progressed |
| Date treated with EIRP | May 2009 |
| EIRP treatment | 5 days with 5 treatments |
| Results Achieved with EIRP | Patient felt improvement in her state on day four of |
| | treatment. |
| | Improvement continued, following treatment. |
| Complications and side effects | There were no adverse effects observed related to the |
| | treatment. |
| Current condition | At 5 month after EIRP treatment, the patient is symptom |
| | free. |

TABLE 10
(Patient #9; canine)

| | |
|---|---|
| Age and sex | Pittbull dog, 37 kg, 10 years |
| Condition or disease | Psoriatic lesions on legs and shoulder. |
| Severity | Dog constantly felt irritation in place of lesions |
| Prior treatment | Corticosteroid therapy gave transitory short-lasting release |
| Results achieved in prior treatment | ineffective - lesions still persisted |
| Date treated with EIRP | January 2009 |
| EIRP treatment | 2 days with 2 treatments |
| Results Achieved with EIRP | No visible improvement after 2 days of treatment. |
| | 30 days - all lesions disappeared. |
| Complications and side effects | There were no adverse effects observed related to the |
| | treatment. |
| Current condition | At 10 months after treatment dog still is free of skin |
| | lesions. |

All examples included in this application are for the purpose of illustration of the invention only and are not intended in any way to limit the scope of the present invention. It will thus be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be falling within the scope of the invention.

Having now fully described the present invention in some detail by way of illustration and example for purposes of clarity of understanding, it will be readily apparent to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. All examples included in this application are for the purpose of illustration of the invention only and are not intended in any way to limit the scope of the present invention. It will thus be readily apparent to one skilled in the art that varying substitutions and modifications may be made to the invention disclosed herein without departing from the scope and spirit of the invention. Thus, it should be understood that although the present invention has been specifically disclosed by preferred embodiments and optional features, modification and variation of the concepts herein disclosed may be resorted to by those skilled in the art, and that such modifications and variations are considered to be within the scope of the present invention.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of ameliorating, treating or preventing disease or disorder associated with a presence of one or more autoantibodies in a circulation of a mammal, comprising, in sequence:
(a) removing one or more of said autoantibodies from the circulation of said mammal; and
(b) administering to said mammal an intravenous immunoglobulin preparation (IVIG) comprising at least 20% immunoglobulin κ2 in an amount sufficient to restore the immune system of said mammal to homeostasis,
wherein the IVIG preparation contains no detectable viral organisms.

2. The method of claim 1, wherein said disease is cancer.

3. The method of claim 1, wherein said disorder is an auto-immune disorder.

4. The method of claims 1-3, wherein said one or more autoantibodies bind to an epitope of at least one anti-angiogenic factor.

5. The method of claim 4, wherein said anti-angiogenic factor is angiostatin.

6. The method of claims 1-5, wherein said autoantibodies are removed from the circulation of said mammal by apheresis.

7. The method of claims 1-6, wherein said apheresis is plasmapheresis.

8. The method of claim 7, wherein said plasmapheresis removes from about 100 ml to about 1000 ml of plasma from the body of said mammal.

9. The method of claims 1-7, wherein said plasmapheresis is carried out over a period of from one to six hours.

10. The method of claims 1-9, wherein said immunoglobulins are administered to said mammal intravenously.

11. The method of claims 1-10, wherein said immunoglobulins are mixed gamma globulins or IgG.

12. The method of claims 1-11, wherein said immunoglobulins are administered to said mammal over a period of from one to ten days.

13. The method of claim 1-12, wherein said immunoglobulins are administered to said mammal in an amount totaling about 2.5 grams to about 200 grams, about 5 grams to about 100 grams, about 5 grams to about 80 grams, or about 10 grams to about 40 grams.

14. The method of claims 1-13, wherein said immunoglobulins are administered to said mammal over a course of from one to five days.

15. The method of claims 1-14, wherein said immunoglobulins are administered to said mammal over a course of five days according to the following schedule:
(a) on Day 2, 0 to 2 grams;
(b) on Day 3, 0 to 4 grams;
(c) on Day 4, 0 to 5 grams;
(d) on Day 5, 0 to 7 grams; and
(e) on Day 6, 0 to 10 grams.

16. The method of claims 1-14, wherein said immunoglobulins are administered to said mammal over a course of five days according to the following schedule:
(a) on Day 2, 1.25 grams;
(b) on Day 3, 2.5 grams;
(c) on Day 4, 0 grams;
(d) on Day 5, 5 grams; and
(e) on Day 6, 10 grams.

17. The method of claims 1-16, wherein said disease or disorder is selected from the group consisting of a neoplastic disease, an autoimmune disease or disorder, a cardiovascular disease, a respiratory disease, a urinary tract disease, a gastrointestinal tract disease, a reproductive disorder, a nervous system disease, a mental disorder, a musculoskeletal system disease, an endocrine disease, a connective tissue disease, a skin disease, a transplantation disease, a disease related to one or more sensory organs, and an infectious disease.

18. The method of claim 17, wherein said neoplastic disease is selected from the group consisting of a carcinoma, a sarcoma, a lymphoma, a leukemia, a germ cell tumor, a lion-brain carcinoma and a blastoma.

19. The method of claim 17, wherein said autoimmune disease or disorder is selected from Lupus erythematosus, Addison's disease, Alopecia areata, Ankylosing spondylitis, Antiphospholipid antibody syndrome, Autoimmune hepatitis, Autoimmune inner ear disease, Bullous pemphigoid, Behçet's disease, Coeliac disease, Chagas disease, Chronic obstructive pulmonary disease, Crohns Disease, Dermatomyositis, Endometriosis, Goodpasture's syndrome, Graves' disease, Guillain-Barré syndrome, Hidradenitis suppurativa, IgA nephropathy, Kawasaki disease, Interstitial cystitis, Idiopathic thrombocytopenic purpura, Morphea, Multiple sclerosis, Pernicious anaemia, Schizophrenia, Psoriasis, Sjögren's syndrome, Scleroderma, Rheumatoid arthritis, Dermatomyositis, Diabetes mellitus type 1 (LADA), Hashimoto's thyroiditis, Addison's disease, Pemphigus vulgaris, Autoimmune haemolytic anaemia, Vasculitis, Vitiligo, and Wegener's granulomatosis.

20. The method of claims 1-19, wherein said mammal is selected from the group consisting of a human, a mouse, a rat, a dog, a cat, a rat, a bovine species, a porcine species, an ovine species and an equine species.

21. The method of claims 1-20, further comprising administering at least one anticoagulant agent to said patient.

22. The method of claim 21, wherein said anticoagulant agent is selected from the group consisting of glucose citrate, heparin, ximelagatran, argatroban, lepirudin, bivalirudin, warfarin, phenindione, acenocoumarol and phenprocoumon.

23. The method of claims 1-22, further comprising administering to said patient at least one antihistamine immediately prior to, during or immediately following administration of said immunoglobulins to said patient.

24. The method of claim 23, wherein said antihistamine is selected from the group consisting of diphenhydramine, loratadine, desloratadine, fexofenadine, meclizine, pheniramine, cetirazine, promethazine, chlorpheniramine, levocetirazine, cimetidine, famotidine, ranitidine, ciproxifan and clobenpropit.

25. The method of claims 1-24, further comprising administering to said patient at least one non-steroidal antiinflammatory agent immediately prior to, during or immediately following administration of said immunoglobulins to said patient.

26. The method of claim 25, wherein said non-steroidal anti-inflammatory agent is selected from the group consisting of aspirin, ibuprofen, naproxen, diclofenac, aceclofenac and licofelone.

27. The method of claim 1, wherein said autoantibodies are removed from the circulation of said mammal by de-glycosylation thereof.

28. The method of claim 27, wherein de-glycosylation of said autoantibodies are achieved by administration of enzymes removing the oligosaccharide fragment of said autoantibodies to the circulation of said mammal

29. The method of claim 28, wherein said enzyme is endoglycosidase, particularly endoglycosidase-S.

30. The method of claims 1-29, wherein the IVIG preparation comprises at least 30% active immunoglobulin κ2, at least 45% active immunoglobulin κ2, or greater than about 50% active immunoglobulin κ2

31. A method of purifying a human intravenous immunoglobulin (IVIG) from a bodily fluid, wherein the resultant IVIG is suitable for therapeutic use, the method comprising the steps of:
(a) removing one or more components of coagulation pathway from the bodily fluid;
(b) adding one or more alcohols to the bodily fluid to remove undesired proteins;
(c) concentrating the bodily fluid under conditions that avoid activation of the complement pathway in the bodily fluid; and
(d) treating the bodily fluid to eliminate one or more active viral and microbial contaminants; and
(e) assaying the activity of the IVIG at least after (d) to obtain a purified IVIG from the plasma protein concentrate, wherein the purified IVIG is a highly effective IVIG for treating one or more disease or disorder in a mammal.

32. The method of claim 31, wherein the activity of the IVIG is assayed after each of (a)-(d).

33. The method of claims 31-32, further comprising adjusting the pH of the plasma protein concentrate to about 5.

34. The method of claims 31-33, further comprising incubating the plasma protein concentrate at a temperature of about 30° C.

35. The method of claim 34, wherein the plasma protein concentrate is incubated at 30° C. for about 2 weeks.

36. The method of claims 31-35, further involving diluting the plasma protein concentrate following the addition of one or more alcohols to the plasma protein concentrate.

37. The method of claim 36, wherein the plasma protein concentrate is diluted to a concentration of less than about 12.5 g/L.

38. The method of claims 31-37, wherein removal of one or more components of the coagulation system comprises the steps of cryoprecipitation and ion exchange chromatography.

39. The method of claims 31-38, wherein the one or more alcohol comprises ethanol.

40. The method of claims 31-39, wherein the concentrating is performed by ultrafilter.

41. The method of claims 31-40, wherein said one or more active viral contaminants comprises one or more enveloped viras or one or more non-enveloped viruses.

42. The method of claims 31-41, wherein said elimination of one or more active viral and microbial contaminants from the plasma protein concentrate comprises one or more filtration steps.

43. Compositions for ameliorating, treating or preventing disease or disorder associated with the presence of one or more autoantibodies in the circulation of a mammal, wherein the compositions contain one or more immunoglobulins, obtained by the method of claims 31 to 42.

44. A method of assessing a state of an immune system in a mammal, comprising:
(i) collecting a urine sample from the mammal;
(ii) loading at least about 100 ml of the urine sample onto an affinity chromatography column;
(iii) washing the column with at least about 3-5 column volumes of a washing buffer, wherein the washing buffer has a pH of at least about 6;
(iv) eluting light chain immunoglobulins from the column using at least about 1-2 column volumes of an eluting buffer, wherein the eluting buffer has a pH of about 2.3-3.5;
(v) quantifying an amount of the light chain immunoglobulins eluted from the column; and
(vi) making an assessment of the state of the immune system in the mammal, wherein the amount of at least about 1 μg of immunoglobulins in the urine sample signifies a presence of an autoimmune disease or disorder in the mammal.

45. A method of assessing a state of an immune system in a mammal, comprising:
(i) collecting a plasma sample from the mammal;
(ii) loading at least about 0.2 ml of the plasma sample onto an affinity chromatography column;
(iii) eluting immunoglobulins κ1 from the column with at least about 1-2 column volumes of an eluting buffer, wherein the eluting buffer has a pH of at least about 5;
(iv) quantifying an amount of immunoglobulins κ1 eluted from the column with the eluting buffer buffer in step (iii);
(v) eluting immunoglobulins κ2 from the column using at least about 1-2 column volumes of an eluting buffer, wherein the eluting buffer has a pH of about 2.3-3.5;
(vi) quantifying an amount of the immunoglobulins κ2 eluted from the column with the eluting buffer; and
(vii) making an assessment of the state of the immune system in the mammal, wherein a presence of the immunoglobulins κ1 in an amount that is less than about 0.05% the amount of the immunoglobulins κ2 signifies a presence of an autoimmune disease or disorder in the mammal.

46. The method of claim 44 or 45, wherein the affinity chromatography column comprises immobilized protein selected from the group consisting of protein A, protein L, protein G, and a combination thereof.
